(19) United States
(12) Patent Application Publication
(10) Pub. No.: US 2017/0128038 A1
(43) Pub. Date: May 11, 2017

(54) ULTRASOUND DIAGNOSTIC DEVICE AND ULTRASOUND SIGNAL PROCESSING METHOD

(71) Applicant: Konica Minolta, Inc., Tokyo (JP)

(72) Inventor: Mineo TSUSHIMA, Souraku-gun Seika-cho (JP)

(21) Appl. No.: 15/340,298

(22) Filed: Nov. 1, 2016

(30) Foreign Application Priority Data
Nov. 6, 2015 (JP) 2015-218241

(51) Int. Cl. A61B 8/08; A61B 8/00; G01S 7/52

(52) U.S. Cl. CPC A61B 8/0841; G01S 7/5209; A61B 8/4483; A61B 8/461; A61B 8/5207; A61B 8/4444; A61B 8/5215

(57) ABSTRACT

Ultrasound diagnostic device including: a transmitter that performs events by transmitting ultrasound focusing inside a subject by using a first transducer group that gradually shifts in an array direction between events; and a receiver that, for each event, generates receive signal sequences for transducers based on ultrasound reflection that the transducers receive in response to the event, that selects a second transducer group whose receive signals based on ultrasound reflection from a puncture needle have high intensity, that, for each event, sets a target area for generating a sub-frame data item inside a virtual area of the subject that receives ultrasound transmitted in the event, and generates the sub-frame data item by performing, for each measurement point in the target area, delay-and-sum processing on receive signal sequences for the second transducer group, and that generates a frame data item by combining sub-frame data items for events.

### CROSS-REFERENCE TO RELATED APPLICATION

This application is based on an application No. 2015-218241 filed in Japan, the contents of which are hereby incorporated by reference.

### BACKGROUND OF THE INVENTION

(1) Field of the Invention

The present disclosure relates to ultrasound diagnostic devices and ultrasound signal processing methods. In particular, the present disclosure relates to beam forming for ultrasound image diagnosis, adapted for the use of a puncture needle.

(2) Description of the Related Art

There exists a medical test called biopsy. In typical biopsy, a puncture needle is inserted into a body of a patient (an examination subject) to remove samples of body tissues and body fluids from the examination subject. Further, anesthesiology, which is practiced by anesthesiologists in intensive care units, pain clinics, etc., also involves the use of puncture needles. In such medical practices, a medical practitioner inserts a puncture needle into the examination subject while checking the position of the puncture needle in ultrasound images that are acquired by using an ultrasound probe (probe) and that show the inside of the examination subject. In inserting the puncture needle into the examination subject, the medical practitioner should be able to specify the puncture needle, particularly the position of the tip of the puncture needle, on a monitor. Thus, there is a demand for ultrasound diagnostic devices producing ultrasound images imaging a puncture needle with high visual perceptibility.

A conventional ultrasound diagnostic device causes transducer elements of a probe to transmit ultrasound towards the inside of the examination subject, in response to which the transducer elements receive, from the inside of the examination subject, ultrasound reflection (echoes) generated due to different body tissues of the examination subject having different levels of acoustic impedance. Based on electric signals acquired based on the ultrasound reflection received, the ultrasound diagnostic device generates ultrasound tomographic images showing the structure of body tissues of the examination subject and displays the ultrasound tomographic images on a monitor.

One method that a conventional ultrasound diagnostic device uses for the generation of signals from ultrasound reflection (for reception beam forming) is delay-and-sum processing. One example of delay-and-sum processing can be found disclosed in pages 42-45 of “Ultrasound Diagnostic Device”, written by Masayasu Itou and Tsuyoshi Mochizuki and published by Corona Publishing Co., Ltd (Aug. 26, 2002). FIG. 29 is a schematic illustrating reception beam forming by a conventional ultrasound diagnostic device. The conventional ultrasound diagnostic device illustrated in FIG. 29 has a probe 201 connected thereto. The probe 201 has a plurality of ultrasound transducer elements (transducer elements) 201a that receive ultrasound reflection from inside the examination subject. Further, the conventional ultrasound diagnostic device has a reception beam former 202. The reception beam former 202 is linked with the transducer elements 201a, and performs amplification, A/D conversion, and delaying (phase adjustment) on electric signals based on ultrasound reflection that the transducer elements 201a receives. Further, the reception beam former 202 multiplies the signals output as a result of such processing by weights (apodization), and outputs sums of the weighted signals as acoustic line signals. When using this reception beam forming method, the ultrasound diagnostic device usually causes the transducer elements 201a to transmit ultrasound (i.e., performs transmission beam forming) so that an ultrasound beam transmitted from the transducer elements 201a focuses at a predetermined depth of the examination subject. Further, the ultrasound diagnostic device sets measurement points P (points for which acoustic line signals are generated) along the central axis of the transmitted ultrasound beam, and calculates delay amounts for the transducer elements 201a with respect to each measurement point P based on the distance between the transducer elements 201a and the measurement point P.

Signal generation efficiency per ultrasound transmission is low with this method, since the ultrasound diagnostic device is only capable of generating, for each transmission of ultrasound, acoustic line signals for only one or a few lines of measurement points P along the central axis of the ultrasound beam. Further, with this method, an acoustic line signal generated for a measurement point P not in the vicinity of a transmission focal point has low resolution and low S/N ratio. Thus, the conventional ultrasound diagnostic device is not capable of producing ultrasound images imaging the inside of the examination subject with high visible perceptibility.

In view of such problems, another reception beam forming method is being proposed that utilizes the so-called synthetic aperture method to yield high resolution, high quality images not only from the vicinity of a transmission focal point but also from outside the vicinity of the transmission focal point. One example of such a reception beam forming method can be found disclosed in pages 395 through 405 of “Virtual Ultrasound Sources in High Resolution Ultrasound Imaging”, S. I. Nikolov and J. A. Jensen, in Proc, SPIE—Progress in Biomedical Optics and Imaging, Vol. 3, 2002. According to this method, delaying is performed taking into consideration both propagation paths of ultrasound and the amount of time required for ultrasound reflection to arrive at transducer elements by travelling along the propagation paths. Thus, this method achieves reception beam forming making use of ultrasound reflection from both the vicinity of the transmission focal point and the outside of the vicinity of the transmission focal point in an ultrasound main transmission area. Thus, for each transmission of ultrasound, acoustic line signals can be generated for the entire ultrasound main irradiation area. In addition, the synthetic aperture method achieves setting a virtual focus on each measurement point by using multiple signals acquired from the measurement point as a result of multiple transmissions of ultrasound. Thus, reception beam forming method utilizing the synthetic aperture method produces ultrasound images that have higher resolution and higher S/N ratio and that image objects with higher visual perceptibility than those produced with the reception beam forming method disclosed in “Ultrasound Diagnostic Device”.

### SUMMARY

### Problems to be Solved

Ultrasound diagnostic devices cause a probe to transmit an ultrasound beam towards the inside of the examination subject and visualize the ultrasound reflection acquired in response. Accordingly, in order for an ultrasound diagnostic device to be able to image a puncture needle being inserted into the examination subject with high visual perceptibility, the ultrasound diagnostic device should be able to accurately generate signals based on both ultrasound reflection from body tissues and ultrasound reflection from the puncture needle.

Here, it should be noted that a puncture needle produces specular ultrasound reflection, whereas body tissues produce diffuse ultrasound reflection. Thus, ultrasound reflection from a puncture needle has high directivity. This means that ultrasound reflection from a puncture needle has strong intensity in a certain direction, which changes depending upon the insertion angle of the puncture needle with respect to the examination subject and the position of the puncture needle relative to the probe. In other words, depending upon conditions such as the angle and the position of the puncture needle relative to the examination subject, ultrasound reflection that a transducer element of a probe receives from the puncture needle may have low intensity, which results in the puncture needle not being imaged with a sufficient level of visual perceptibility.

In particular, when employing the reception beam forming method disclosed in “Virtual Ultrasound Sources in High Resolution Ultrasound Imaging”, which utilizes the synthetic aperture method, measurement points may be set over the entire ultrasound main irradiation area. Due to this, depending upon the position of the puncture needle relative to the measurement point for which a signal is to be generated and transducer elements whose ultrasound reflection is to be used in signal generation, a generated signal may barely show ultrasound reflection from the puncture needle. This results in the puncture needle being imaged with low visual perceptibility.

In view of these technical problems, the present disclosure aims to provide an ultrasound signal processing method and an ultrasound diagnostic device producing ultrasound images imaging a puncture needle with high visible perceptibility, with reception beam forming utilizing the synthetic aperture method.

### Means for Solving the Problems

One aspect of the present disclosure is an ultrasound diagnostic device to which an ultrasound probe having a plurality of transducer elements arrayed along an array direction is connectable, wherein the ultrasound diagnostic device: (i) repeatedly performs transmission events, each by selecting a first group of transducer elements from among the plurality of transducer elements and causing the first group to transmit ultrasound towards an examination subject having a puncture needle inserted therein; (ii) for each of the transmission events, generates a sub-frame data item based on ultrasound reflection received in response to the transmission event, the sub-frame data item including acoustic line signals; and (iii) combines sub-frame data items for the transmission events to generate a frame data item, the frame data item including combined acoustic line signals each being an aggregate of ones of the acoustic line signals, and the ultrasound diagnostic device includes an ultrasound signal processing circuit operating as: a transmitter that performs each of the transmission events by selecting the first group and causing the first group to transmit ultrasound focusing inside the examination subject, the transmitter gradually shifting the first group in the array direction between transmission events; and a receiver that: (i) for each of the transmission events, selects at least some of the plurality of transducer elements and generates a sequence of receive signals for each of the selected transducer elements based on ultrasound reflection that the selected transducer element has received from the examination subject in response to the transmission event; (ii) selects a second group of transducer elements, the transducer elements in the second group being ones among the plurality of transducer elements whose receive signals based on ultrasound reflection from the puncture needle have high intensity; (iii) for each of the transmission events, sets a target area for generating the sub-frame data item inside a virtual area of the examination subject that receives ultrasound transmitted in the transmission event, and generates the sub-frame data item by performing, for each of a plurality of measurement points in the target area, delay-and-sum processing on receive signals, for the transducer elements in the second group, based on ultrasound reflection from the measurement point; and (iv) generates the frame data item by combining the sub-frame data items for the transmission events.

### BRIEF DESCRIPTION OF THE DRAWINGS

These and the other objects, advantages and features of the technology pertaining to the present disclosure will become apparent from the following description thereof taken in conjunction with the accompanying drawings, which illustrate specific embodiments of the technology pertaining to the present disclosure.

In the drawings:

FIG. 1 illustrates functional blocks of an ultrasound diagnostic system 1000 including an ultrasound diagnostic device 100 pertaining to embodiment 1;

FIG. 2 is a schematic illustrating a propagation path of ultrasound transmitted by a transmitter 107;

FIG. 3 illustrates functional blocks of a transmission beam former 106;

FIG. 4 illustrates functional blocks of a reception beam former 109;

FIG. 5 is a schematic illustrating, based on one transmission event, the relationship between a transmission transducer element array Tx, an ultrasound main irradiation area Ax, a target area Bx, a reception transducer element array Rx, and a reception apodization;

Each of FIGS. 6A and 6B is a schematic illustrating an overview of calculation of ultrasound propagation path, performed by a delay processor 10921 of a delay-and-sum calculator 1092;

Each of FIGS. 7A and 7B is a schematic illustrating generation of an acoustic line signal for a measurement point Pij, performed by the delay-and-sum calculator 1092;

FIG. 8 is a schematic illustrating combining of acoustic line signal sub-frame data items, performed by a sub-frame combiner 10931;

Each of FIGS. 9A and 9B is a schematic illustrating amplification performed by a synthesizer 1093;

FIG. 10 illustrates functional blocks of an in-puncture reception transducer element array selector 113;

FIG. 11 is a schematic illustrating processing performed by the in-puncture reception transducer element array selector 113;

Each of FIGS. 12A and 12B is a schematic illustrating processing performed by the in-puncture reception transducer element array selector 113;

Each of FIGS. 13A through 13D is a schematic illustrating processing performed by the in-puncture reception transducer element array selector 113;

Each of FIGS. 14A through 14D is a schematic illustrating processing performed by the in-puncture reception transducer element array selector 113;

FIG. 15 is a flowchart illustrating beam forming performed by the ultrasound diagnostic device 100;

FIG. 16 is a flowchart illustrating selection of a reception transducer element array Rx, performed by the in-puncture reception transducer element array selector 113;

FIG. 17 is a flowchart illustrating generation of an acoustic line signal for a measurement point Pij, performed by the delay-and-sum calculator 1092;

FIG. 18 illustrates functional blocks of an ultrasound diagnostic system 1000A including an ultrasound diagnostic device 100A pertaining to embodiment 2;

FIG. 19 illustrates functional blocks of an in-puncture reception transducer element array selector 113A;

Each of FIGS. 20A through 20C is a schematic illustrating processing performed by the in-puncture reception transducer element array selector 113A, with an acoustic line signal sub-frame data item illustrated over a corresponding position within an examination subject;

FIG. 21 is a flowchart illustrating selection of a reception transducer element array Rx, performed by the in-puncture reception transducer element array selector 113A;

FIG. 22 illustrates functional blocks of an ultrasound diagnostic system 1000B including an ultrasound diagnostic device 100B pertaining to embodiment 3;

FIG. 23 illustrates functional blocks of an in-puncture reception transducer element array selector 113B;

Each of FIGS. 24A and 24B is a schematic illustrating processing by the in-puncture reception transducer element array selector 113B;

FIG. 25 is a flowchart illustrating selection of a reception transducer element array Rx, performed by the in-puncture reception transducer element array selector 113B;

FIG. 26 illustrates functional blocks of an ultrasound diagnostic system 1000C including an ultrasound diagnostic device 100C pertaining to embodiment 4;

FIG. 27 illustrates functional blocks of an in-puncture reception transducer element array selector 113C;

FIG. 28 is a flowchart illustrating selection of a reception transducer element array Rx, performed by the in-puncture reception transducer element array selector 113C; and

FIG. 29 is a schematic illustrating reception beam forming by a conventional ultrasound diagnostic device.

### DESCRIPTION OF PREFERRED EMBODIMENTS

The following describes embodiments of the technology pertaining to the present disclosure.

### Embodiment 1

### <Ultrasound Diagnostic System 1000>

### 1. Overview of Structure

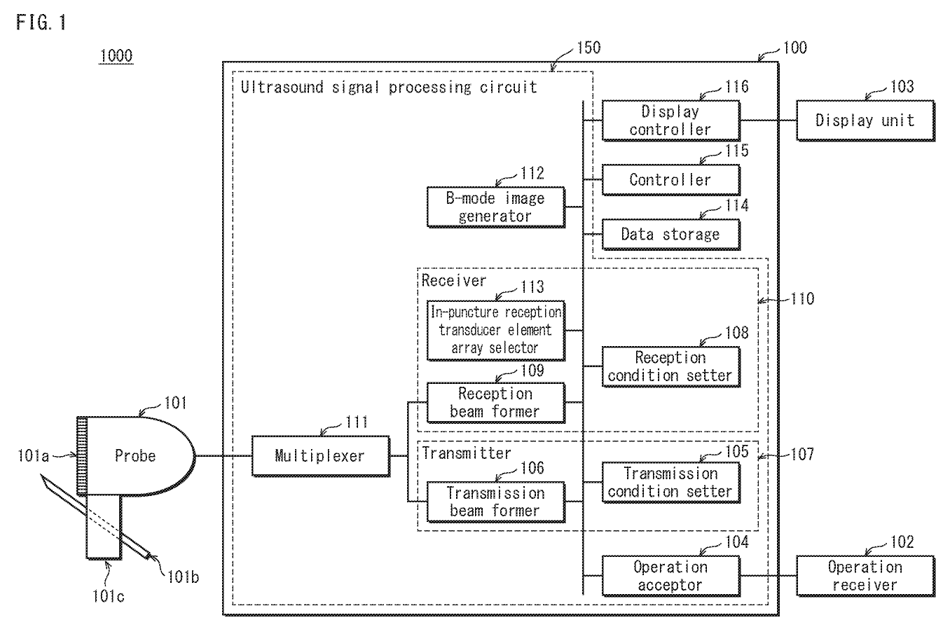

The following describes an ultrasound diagnostic system 1000 with reference to the accompanying drawings. The ultrasound diagnostic system 1000 includes an ultrasound diagnostic device 100 pertaining to embodiment 1. FIG. 1 illustrates functional blocks of the ultrasound diagnostic system 1000. As illustrated in FIG. 1, the ultrasound diagnostic system 1000 includes, in addition to the ultrasound diagnostic device 100, an ultrasound probe 101 (probe 101), an operation receiver 102, and a display unit 103. The probe 101 has a plurality of transducer elements 101a arrayed at a front tip surface thereof. The transducer elements 101a are capable of transmitting ultrasound towards an examination subject and receiving ultrasound reflection from the examination subject. The ultrasound diagnostic device 100 causes the probe 101 to perform transmission and reception of ultrasound, and generates ultrasound images based on signals output from the probe 101. The operation receiver 102 receives input operations performed by an examiner. The display unit 103 displays ultrasound images on a screen. Each of the probe 101, the operation receiver 102, and the display unit 103 can be connected to the ultrasound diagnostic device 100 or disconnected from the ultrasound diagnostic device 100. FIG. 1 illustrates the ultrasound diagnostic device 100 with the probe 101, the operation receiver 102, and the display unit 103 connected thereto. Alternatively, the probe 101, the operation receiver 102, and the display unit 103 may be incorporated into the ultrasound diagnostic device 100.

The following describes each of the probe 101, the operation receiver 102, and the display unit 103, which are external components connectable to the ultrasound diagnostic device 100.

### 2. Probe 101

The probe 101 has transducer elements 101a that are, for example, arrayed along one direction (array direction). The probe 101 receives a pulsar electric signal (transmission signal) from a later-described transmission beam former 106 of the ultrasound diagnostic device 100, and converts the pulsar electric signal into ultrasound pulses. Here, while the probe 101 is described as converting a pulsar signal into ultrasound pulses for the sake of simplicity, the input and output of the probe 101 are not limited to being pulsar in such a manner. The probe 101, with an outer surface thereof where the transducer elements 101a are arranged in contact with a skin surface of the examination subject, transmits an ultrasound beam towards the examination subject. Here, an ultrasound beam is considered as an aggregate of ultrasound transmitted from a plurality of transducer elements 101a. Further, the probe 101 receives ultrasound reflection from the examination subject by using a plurality of transducer elements 101a. The transducer elements 101a receiving ultrasound reflection convert the ultrasound reflection into electric signals, and the probe 101 supplies these electric signals to a later-described reception beam former 109 of the ultrasound diagnostic device 100.

Further, the probe 101 has a puncture guide 101c attached thereto. The puncture guide 101c holds a puncture needle 101b. Specifically, the puncture guide 101c has an undepicted guide member that, when the probe 101 is put in contact with the skin surface of the examination subject, guides the puncture needle 101b into the examination subject at a predetermined insertion angle. Further, a configuration may be made such that the angle of the puncture guide 101c relative to the probe 101 is changeable, which enables adjusting the insertion angle of the puncture needle 101b with respect to the examination subject.

### 3. Operation Receiver 102

The operation receiver 102 receives an input operation that the examiner performs in operating the ultrasound diagnostic device 100, and outputs the input operation to be received by a later-described controller 115 of the ultrasound diagnostic device 100 via a later described operation acceptor 104 of the ultrasound diagnostic device 100. The input operation may be, for example, an input operation for changing a setting of the ultrasound diagnostic device 100 or an input operation for controlling the ultrasound diagnostic device 100. Further, a configuration may be made, for example, such that the operation receiver 102 is capable of receiving, from the examiner, manual input for setting the insertion angle of the puncture needle 101b with respect to body tissues of the examination subject and the position of the puncture needle 101b relative to the transducer elements 101, as described later in detail in embodiment 4.

Note that the combination of the operation receiver 102 and the display unit 103 may be implemented by using a touch panel. With this configuration, the examiner can change settings of the ultrasound diagnostic device 100 and control the ultrasound diagnostic device 100 by performing touch input, drag input, etc., by using one or more operation keys displayed on the display unit 103. Alternatively, the operation receiver 102 may be, for example, a keyboard having keys that the examiner can use to perform various operations, or an operation panel having one or more buttons, one or more levers, and/or the like that the examiner can use to perform various operations. Alternatively, the operation receiver 102 may be, for example, a trackball, a mouse, or a flat pad that the examiner can use to control a cursor displayed on the display unit 103. Further, the operation receiver 102 may be a combination of two or more of such devices. Also, a plurality of operation receivers 102 may be provided, each being one of such devices.

### 4. Display Unit 103

The display unit 103 is an image display device, and has a screen for displaying images output from a later-described display controller 116 of the ultrasound diagnostic device 100. The display unit 103 may be, for example, a liquid crystal display (LCD), a cathode ray tube (CRT), or an organic electroluminescence (EL) display.

### <Overview of Structure of Ultrasound Diagnostic Device 100>

The following describes the ultrasound diagnostic device 100 pertaining to embodiment 1.

The ultrasound diagnostic device 100 includes: a transmission condition setter 105; a transmission beam former 106; a reception condition setter 108; a reception beam former 109; a multiplexer 111; and an in-puncture reception transducer element array selector 113. The transmission condition setter 105 generates information indicating a group of transducer elements 101a that are to perform ultrasound transmission, and outputs the information to the transmission beam former 106. In the following, the transducer elements 101a that perform ultrasound transmission are referred to as transmission transducer elements 101a, and the group of the transmission transducer elements 101a is referred to as a transmission transducer element array Tx. The transmission beam former 106 determines when a high voltage triggering ultrasound transmission is to be applied to the transmission transducer elements 101a. The reception condition setter 108 generates information indicating a group of transducer elements 101a whose receive signals are to be used for the generation of acoustic line signals, and outputs the information to the reception beam former 109. In the following, the transducer elements 101a whose receive signals are used for the generation of acoustic line signals are referred to as reception transducer elements 101a, and the group of the reception transducer elements 101a is referred to as a reception transducer element array Rx. The multiplexer 111 is capable of connecting to either the transmission transducer elements 101a or the reception transducer elements 101a, and provides the transducer elements 101a to which it is connected with input and receives output from the transducer elements 101a to which it is connected. The in-puncture reception transducer element array selector 113 selects, as a reception transducer element array Rx, a group of transducer elements 101a whose receive signals based on ultrasound reflection from the puncture needle 101b have relatively high intensity.

The ultrasound diagnostic device 100 further includes a B-mode image generator 112. The B-mode image generator 112 generates ultrasound images (B-mode images) one after another by using acoustic line signals that the reception beam former 109 generates and outputs based on ultrasound transmission by the transmission beam former 106.

The ultrasound diagnostic device 100 further includes: an operation acceptor 104; a data storage 114; a display controller 116; and a controller 115. The operation acceptor 104 receives, from the operation receiver 102, information related to the manual input operation that the examiner has performed on the operation receiver 102. The data storage 114 stores, for example, receive signals and acoustic line signals output from the reception beam former 109, and B-mode images output from the B-mode image generator 112. The display controller 116 forms display images and causes the display unit 103 to display the display images. The controller 115 controls the components of the ultrasound diagnostic device 100.

Among the components of the ultrasound diagnostic device 100, the operation acceptor 104, the transmission condition setter 105, the transmission beam former 106, the reception condition setter 108, the reception beam former 109, the multiplexer 111, the B-mode image generator 112, and the in-puncture reception transducer element array selector 113 form an ultrasound signal processing circuit 150. Further, the transmission condition setter 105 and the transmission beam former 106 form a transmitter 107, and the reception condition setter 108, the reception beam former 109, and the in-puncture reception transducer element array selector 113 form a receiver 110.

The controller 115, the display controller 116, and the components of the ultrasound signal processing circuit 150 are each implemented by using, for example, a hardware circuit such as a field-programmable gate array (FPGA) or an application-specific integrated circuit (ASIC). Alternatively, such components may each be implemented by using a combination of software and a programmable device such as a central processing unit (CPU), a General-purpose computing on graphics processing unit (GPGPU), or any processor. Such components may each be implemented as one circuit component, or as an aggregate of a plurality of circuit components. Further, a plurality of such components may be implemented by using one circuit component, or as an aggregate of a plurality of circuit components.

The data storage 114 is a computer-readable recording medium. For example, the data storage 114 may be implemented by using a flexible disk, a hard disk, an MO, a DVD, a DVD-RAM, or a semiconductor memory. Alternatively, the data storage 114 may be an external storage device connected to the ultrasound diagnostic device 100.

Note that the ultrasound diagnostic device 100 need not have the structure illustrated in FIG. 1. For example, the ultrasound diagnostic device 100 may not include the multiplexer 111. Further, the probe 101 may include a part or the entirety of, for example, at least one of the transmission beam former 106 and the reception beam former 109.

### <Structures of Components of Ultrasound Diagnostic Device 100>

The ultrasound diagnostic device 100 is characterized for including the transmitter 107 and the receiver 110. The transmitter 107 causes transmission transducer elements 101a to perform ultrasound transmission. The receiver 110 performs computation on electric signals that the probe 101 acquires through the reception of ultrasound reflection, and generates acoustic line signals used for forming ultrasound images. Due to this, the following description focuses on the structures and functions of the components included in the transmitter 107 and the receiver 110. Note that components other than those included in the transmitter 107 and the receiver 110 may have structures and functions similar to those in conventional ultrasound diagnostic devices. In other words, the ultrasound diagnostic device 100 may be implemented by replacing a transmitter and a receiver of a conventional ultrasound diagnostic device with the transmitter 107 and the receiver 110 pertaining to the present embodiment, respectively.

### 1. Transmitter 107

The transmitter 107 is connected to the probe 101, via the multiplexer 111. The transmitter 107 determines when a high voltage pulse is to be applied to each of some of the transducer elements 101a of the probe 101 (i.e., each of the transmission transducer elements 101a) to trigger ultrasound transmission from the probe 101. The transmitter 107 repeatedly performs transmissions of ultrasound while gradually shifting the transmission transducer element array Tx in the array direction each time. Consequently, all transducer elements 101a of the probe 101 perform ultrasound transmission. In the following, a single transmission of ultrasound that the transmitter 107 performs by using one transmission transducer element array Tx is referred to as a transmission event.

The transmitter 107 includes the transmission condition setter 105 and the transmission beam former 106.

### 1.1. Transmission Condition Setter 105

For each transmission event, the transmission condition setter 105 supplies the transmission beam former 106 with a transmission control signal. The transmission control signal causes the transmission transducer elements 101a for the transmission event to transmit an ultrasound beam focusing at a transmission focal point FP set at a predetermined depth (focal depth) in the examination subject. A transmission control signal for a transmission event includes the following information for example: (i) information indicating positions of the transmission transducer elements 101a for the transmission event among the transducer elements 101a of the probe 101; (ii) information indicating the position of the transmission focal point FP for the transmission event; and (iii) information indicating a pulse width of ultrasound pulse to be transmitted.

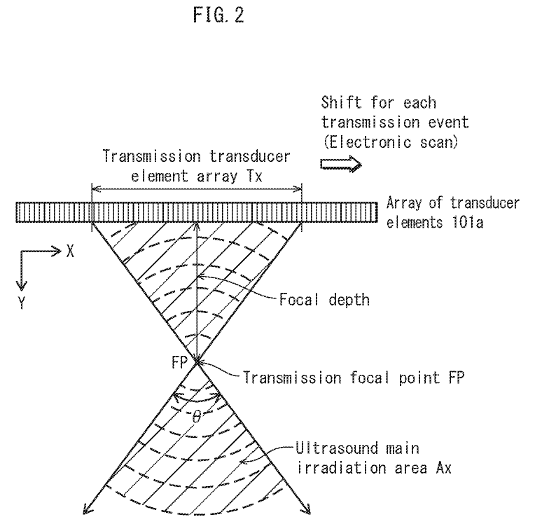

FIG. 2 is a schematic illustrating a propagation path of ultrasound that the transmitter 107 causes the transmission transducer elements 101a to transmit. FIG. 2 illustrates a transmission transducer element array Tx for one transmission event, which is a group of transducer elements 101a that contribute to the transmission event. For example, supposing that the probe 101 has one hundred and ninety two (192) transducer elements 101a in total, the number of transmission transducer elements 101a included in each transmission transducer element array Tx may be twenty (20) to one hundred (100).

Further, the transmission beam former 106 controls when the transmission transducer elements 101a perform ultrasound transmission such that the closer a transmission transducer element 101a is to the center position of the transmission transducer element array Tx in the array direction, the later the transmission transducer element 101a transmits ultrasound in the transmission event. Consequently, the wavefront of ultrasound transmitted from the transmission transducer elements 101a focuses at one point at the focal depth, and thus, the ultrasound beam transmitted from the transmission transducer element array Tx focuses at the transmission focal point FP. Note that the focal depth (i.e., the depth of the transmission focal point FP) can be set as desired or required.

After focusing at the transmission focal point FP, the wavefront of the transmitted ultrasound spreads out as before focusing at the transmission focal point FP. Thus, the transmitted ultrasound mainly propagates through an hourglass-shaped area which has a base defined by the transmission transducer element array Tx and which is partitioned from other areas inside the examination subject by two straight lines intersecting at the transmission focal point FP. Specifically, the ultrasound transmitted from the transmission transducer element array Tx propagates in the following manner. As the ultrasound advances deeper inside the examination subject from the transmission transducer element array Tx, the width thereof (length along X axis in FIG. 2) gradually decreases until reaching the minimum width at the transmission focal point FP. Then, as the ultrasound advances deeper beyond the transmission focal point FP (i.e., as the ultrasound advances in the downward direction in FIG. 2), the width thereof increases (i.e., the ultrasound spreads out). In the following, this hourglass-shaped area described above (indicated by diagonal hatching in FIG. 2) is referred to as an ultrasound main irradiation area Ax.

Further, the F number of transmitted ultrasound (i.e., the ratio of the depth of the transmission focal point FP to the length of the transmission transducer element array Tx) set to the transmission beam former 103 may for example be greater than or equal two and smaller than or equal to four. With this F number, the angle θ at which the two straight lines that define the boundary of the ultrasound main irradiation area Ax intersect at the transmission focal point FP is approximately greater than or equal to 14° and smaller than or equal to 28°.

Note that in the present disclosure, a focused ultrasound beam generated by ultrasound transmission need not focus at a single point as described above, as long as the area that the ultrasound beam irradiates decreases starting from the point of transmission to reach a minimum at the focal depth. When the focused ultrasound beam does not focus at a single point, the transmission focal point FP is the center position of the ultrasound beam at the focal depth.

### 1.2 Transmission Beam Former 106

The transmission beam former 106 is connected to the probe 101, via the multiplexer 111. The transmission beam former 106 receives the transmission control signal from the transmission condition setter 105, and based on the transmission control signal, outputs a high voltage to each transmission transducer element 101a to trigger ultrasound transmission from the probe 101.

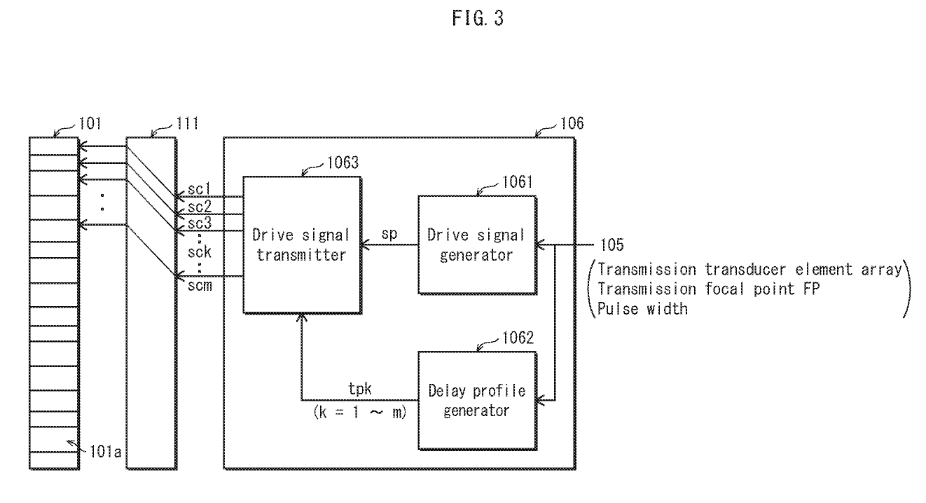

FIG. 3 is a functional block diagram illustrating the structure of the transmission beam former 106. As illustrated in FIG. 3, the transmission beam former 106 includes: a drive signal generator 1061; a delay profile generator 1062; and a drive signal transmitter 1063.

### (1) Drive Signal Generator 1061

The drive signal generator 1061 is a circuit that, based on information in the transmission control signal indicating a transmission transducer element array Tx and a pulse width, generates a pulse signal sp for causing some or all of the transducer elements 101a of the probe 101 (i.e., the transmission transducer element array Tx) to transmit an ultrasound beam.

### (2) Delay Profile Generator 1062

The delay profile generator 1062 is a circuit that, based on information in the transmission control signal indicating a transmission transducer element array Tx and a position of a transmission focal point FP, specifies and outputs a delay time tpk for each transmission transducer element 101a (k denotes a natural number between one and m, inclusive, and m denotes a natural number indicating the total number of transmission transducer elements 101a for a transmission event). The delay time tpk for a transmission transducer element 101a specifies when the transmission transducer element 101a performs ultrasound transmission. Thus, the delay time tpk delays ultrasound transmission by a corresponding transmission transducer element 101a, which results in the generation of a focused ultrasound beam.

### (3) Drive Signal Transmitter 1063

The drive signal transmitter 1063 is a circuit that performs, for each transmission transducer element 101a, transmission processing of supplying a transmission signal sck to the transmission transducer element 101a based on the pulse signal sp from the drive signal generator 1061 and the delay time tpk from the delay profile generator 1062. A transmission signal sck supplied to a transmission transducer element 101a causes the transmission transducer element 101a to perform ultrasound transmission. Note that the transmission beam former 106 is connected to the transmission transducer element array Tx via the multiplexer 111.

The transmission beam former 106, based on transmission control signals from the transmission condition setter 105, repeatedly performs transmission events while gradually shifting the transmission transducer element array Tx in the array direction each time. Consequently, all transducer elements 101a of the probe 101 perform ultrasound transmission. Further, the transmission beam former 106, by repeatedly performing transmission events while gradually shifting the transmission transducer element array Tx in the array direction each time, also gradually shifts the transmission focal point FP in the array direction. Thus, the transmission beam former 106 causes transmitted ultrasound to propagate over the entirety of an examination subject area of the examination subject.

### 2. Receiver 110

The receiver 110 generates acoustic line signals based on electric signals that a plurality of transducer elements 101a have acquired based on ultrasound reflection received by the probe 101. As illustrated in FIG. 1, the receiver 110 includes: the reception condition setter 108; the reception beam former 109; and the in-puncture reception transducer element array selector 113.

The following describes the structure of the components of the receiver 110.

### 2.1. Reception Condition Setter 108

For each transmission event, the reception condition setter 108 generates a reception control signal and supplies the reception beam former 109 with the reception control signal. The reception control signal causes the reception beam former 109 to generate acoustic line signals based on electric signals that a plurality of transducer elements 101a acquire based on ultrasound reflection received by the probe 101 in response to the transmission event. For example, the reception control signal includes information indicating a reception transducer element array Rx and information indicating a target area Bx for which acoustic line signals are to be generated. In addition, the reception control signal may include information indicating a reception apodization.

### a) Setting of Target Area Bx

For each transmission event, the reception condition setter 108 acquires information indicating a transmission transducer element array Tx and information indicating the position of a transmission focal point FP from the transmission condition setter 105. Using such information, the reception condition setter 108 sets a target area Bx for the transmission event. In the present disclosure, a target area Bx is a signal area that is set for each transmission event and that includes a plurality of measurement points Pij, for each of which an acoustic line signal is to be generated. In other words, for each transmission event, acoustic line signals are generated for measurement points Pij included in a target area Bx. Thus, a target area Bx is a group of measurement points Pij for which acoustic line signals are to be generated, and is set for each transmission event for the sake of calculation.

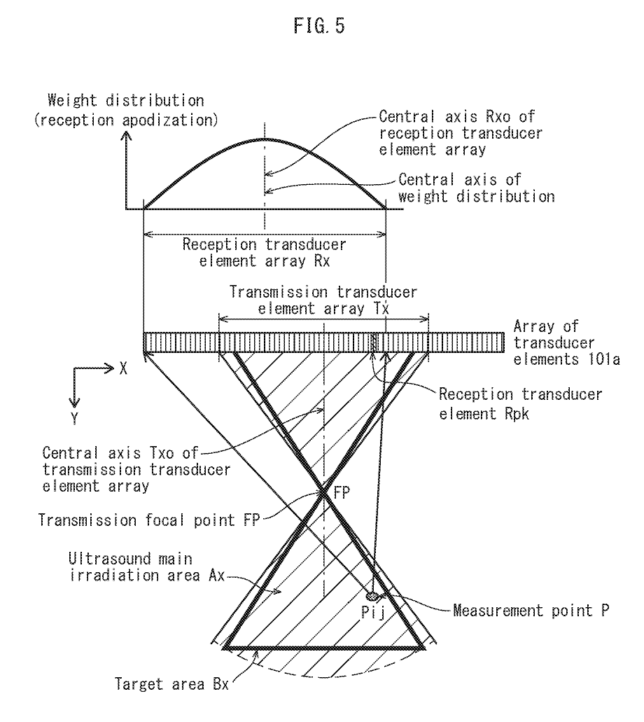

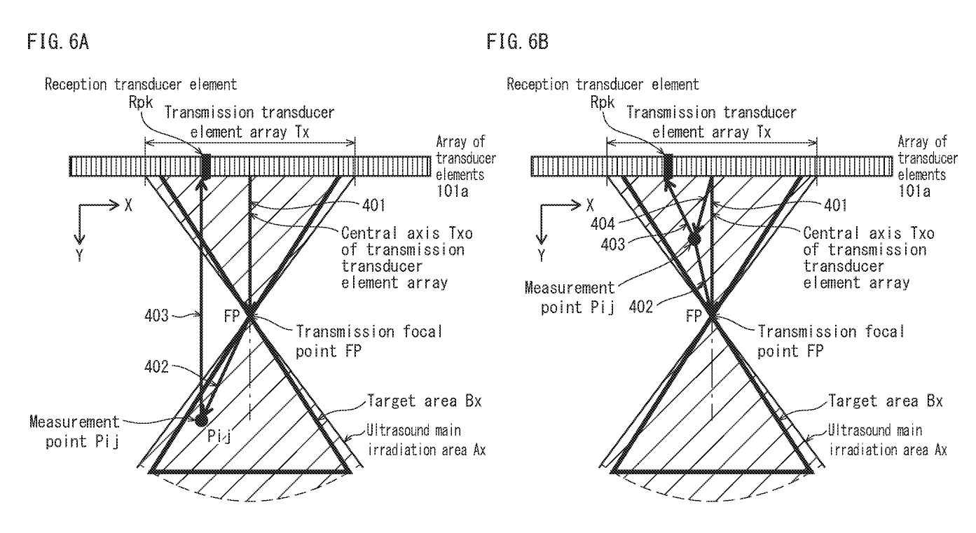

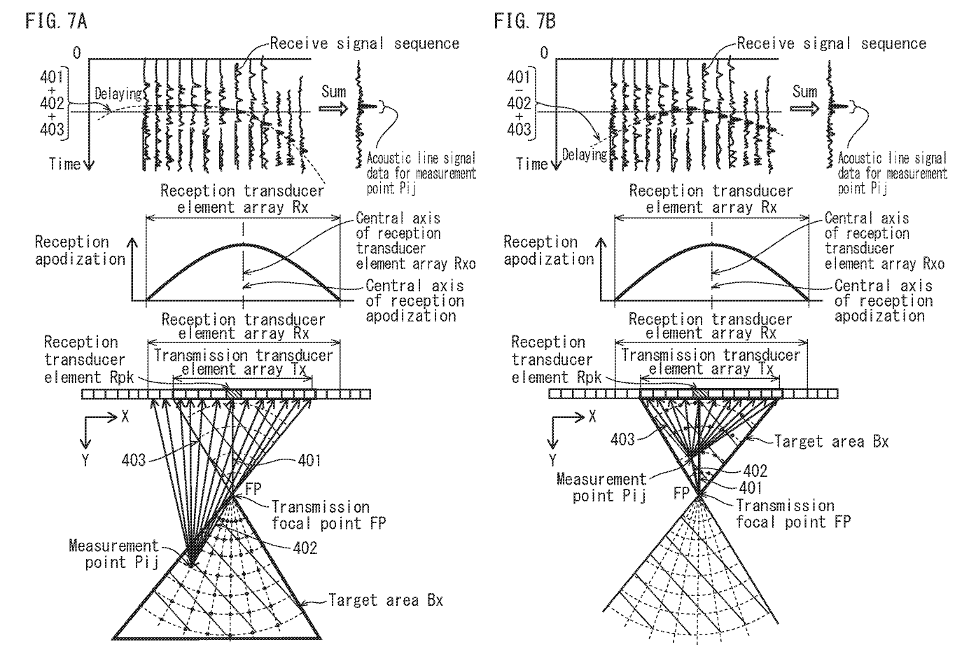

FIG. 5 is a schematic illustrating, based on one transmission event, the relationship between a transmission transducer element array Tx, an ultrasound main irradiation area Ax, a target area Bx, a reception transducer element array Rx, and a reception apodization. As illustrated in FIG. 5, in the present disclosure, a target area Bx is an hourglass-shaped area that is internally tangent to two straight lines passing through the transmission focal point FP and different ends of the transmission transducer element array Tx. As such, the target area Bx is substantially equivalent to the ultrasound main irradiation area Ax.

### b) Setting of Reception Transducer Element Array Rx

The reception condition setter 108 also specifies a reception transducer element array Rx. In the present disclosure, a reception transducer element array Rx is, as illustrated in FIG. 5, a group of transducer elements 101a (reception transducer elements 101a) whose sequences of receive signals are used in delay-and-sum processing for generating acoustic line signals for measurement points Pij.

Specifically, when receiving information indicating a group of transducer elements 101a suitable for generation of acoustic line signals from the puncture needle 101b from the in-puncture reception transducer element array selector 113, the reception condition setter 108 sets the group of transducer elements 101a as a reception transducer element array Rx. Meanwhile, when not receiving information indicating a group of transducer elements 101a suitable for generation of acoustic line signals from the puncture needle 101b from the in-puncture reception transducer element array 113, the reception condition setter 108 sets a reception transducer element array Rx according to a predetermined method. For example, the predetermined method may be (i) a method of setting, for each transmission event, a reception transducer element array Rx such that the center position of the reception transducer element array Rx in the array direction matches the center position of the transmission transducer element array Tx in the array direction, or (ii) a method of setting, for each measurement point Pij, a reception transducer element array Rx such that the center position of the reception transducer element array Rx in the array direction matches the position of the measurement point Pij in the array direction. Here, when setting a reception transducer element array Rx according to a predetermined method as described above, it is preferable that the reception condition setter 108 set the reception transducer element array Rx such that the reception transducer element array Rx has a greater array-direction length than the transmission transducer element array Tx for a corresponding transmission event. For example, the number of transducer elements 101a included in the reception transducer element array Rx may be 32, 64, 96, 128, 192, and so on.

### c) Setting of Reception Apodization

In addition to setting a reception transducer element array Rx, the reception condition setter 108 may also set a reception apodization for the reception transducer element array Rx. In the present disclosure, a reception apodization is information indicating a distribution of weights to be used in delay-and-sum processing. Specifically, the reception apodization is a numerical sequence composed of weight coefficients that are to be applied to sequences of receive signals for the reception transducer elements 101a included in the reception transducer element array Rx. Further, the reception apodization is set so that the maximum weight is set with respect to the reception transducer element 101a located at the center position of the reception transducer element array Rx in the array direction. Thus, as illustrated in FIG. 5, the reception apodization is set so that the central axis of the reception apodization in the array direction matches the central axis Rxo of the receive transducer element array Rx in the array direction. The reception apodization may indicate a weight distribution of any shape, including but not limited to a hamming window, a hanning window, and a rectangular window.

### d) Other Matters

The reception condition setter 108 generates a reception control signal for each transmission event performed. Due to this, the number of times the generation of the reception control signal is performed is equal to the number of transmission events performed. Note that the reception condition setter 108 may perform the generation of reception control signals for transmission events such that a reception control signal is generated each time a transmission event is performed, or alternatively, such that reception control signals for all transmission events are generated at once upon completion of the transmission events.

In the present embodiment, the target area Bx is set to have a shape similar to the shape of the ultrasound main irradiation area Ax. Thus, the target area Bx is an hourglass-shaped area whose base is set along the surface of the examination subject that is in contact with the transmission transducer element array Tx. Setting such a target area Bx enables distributing measurement points over substantially the entire ultrasound main irradiation area Ax, and thereby improves the efficiency of signal generation for each transmission event.

However, the target area Bx need not have an hourglass shape and may have other shapes. For example, when a configuration is made such that transmission transducer elements transmit parallel ultrasound waves, the target area Bx may be configured as a rectangular-shaped area whose base is set along the surface of the examination subject that is in contact with the transmission transducer element array Tx. This configuration also enables distributing measurement points over substantially the entire ultrasound main irradiation area Ax, and thereby improves the efficiency of signal generation for each transmission event.

The reception condition setter 108 outputs the reception control signals to the reception beam former 109.

### 2.2 Reception Beam Former 109

The reception beam former 109 generates an acoustic line signal sub-frame data item dsl for each transmission event, and generates a combined acoustic line signal frame data item ds by combining acoustic line signal sub-frame data items dsl for transmission events. Specifically, for each transmission event, the reception beam former 109 generates an acoustic line signal sub-frame data item dsl by generating an acoustic line signal for each measurement point Pij in the target area Bx. As described above, the target area Bx is set for each transmission event, and the target area Bx for a given transmission event is included in the ultrasound main irradiation area Ax for the transmission event. Further, the reception beam former 109 generates acoustic line signals based on ultrasound reflection that a plurality of transducer elements 101a receive in response to each transmission event.

Specifically, the reception beam former 109 generates, for each transmission event having been performed, acoustic line signals based on electric signals acquired by a plurality of transducer elements 101a based on ultrasound reflection received by the probe 101 in response to the transmission event. In the present disclosure, an acoustic line signal is generated for each measurement point Pij, through delay-and-sum processing of receive signals corresponding to the measurement point Pij.

Further, the reception beam former 109 specifies, as an acoustic line signal sub-frame data item dsl for the transmission event, a set of acoustic lines signals each corresponding to a different one of the measurement points Pij included in the target area Bx for the transmission event. By repeating this process for each transmission event, the reception beam former 109 generates an acoustic line signal sub-frame data item dsl for each transmission event.

Here, note that 1 denotes a natural number between 1 and n, and n denotes the total number of transmission events that are performed. The reception beam former 109 outputs the acoustic line signal sub-frame data items dsl for the transmission events to be stored in the data storage 114.

Further, the reception beam former 109 generates a combined acoustic line signal frame data item ds by combining the acoustic line signal sub-frame data items dsl for the different transmission events. The reception beam former 109 outputs the combined acoustic line signal frame data item ds to be stored in the data storage 114.

Further, the reception beam former 109 generates a sequence of combined acoustic line signal frame data items ds by repeatedly performing the generation of the combined acoustic line signal frame data item ds. The reception beam former 109 outputs the sequence of combined acoustic line signal frame data items ds to be stored in the data storage 114.

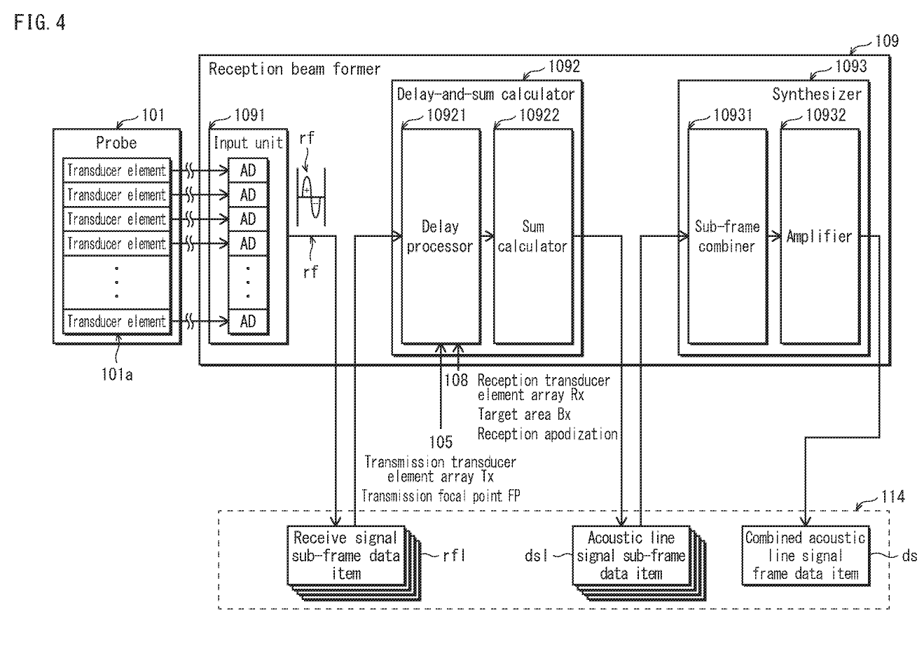

FIG. 4 illustrates functional blocks of the reception beam former 109. The reception beam former 109 includes: an input unit 1091; a delay-and-sum calculator 1092; and a synthesizer 1093.

### (1) Input Unit 1091

The input unit 1091 is connected to the probe 101, via the multiplexer 111. The input unit 1091 is a circuit that generates receive signal sequences (RF signals) based on ultrasound reflection that the probe 101 receives. Specifically, each sequence of receive signals that the input unit 1091 generates corresponds to one transducer element 101a. Specifically, the input unit 1091 generates sequence of receive signals for a transducer element 101a by performing A/D conversion on an electric signal that the transducer element 101a has generated by converting ultrasound reflection received in response to a transmission event. Thus, an RF signal is a digital signal that is a sequence of signals (receive signals rf) along the ultrasound transmission direction (depth direction of examination subject).

For each transmission event, the input unit 1091 generates a sequence of receive signals rf for each of a plurality of transducer elements 101a used to receive ultrasound reflection in response to the transmission event. In the following, the transducer elements 101a that are used to receive ultrasound reflection in response to a transmission event are referred to as receive transducer elements 101a, and the group of the receive transducer elements 101a is referred to as a receive transducer element array. Further, the receive transducer element array is selected for each transmission event. Specifically, the receive transducer element array is a group of some or all of the transducer elements 101a of the probe 101, and is selected for each transmission event based on an instruction from the controller 115. Further, the reception beam former 109 is connected to the receive transducer element array, via the multiplexer 111. Here, it is preferable that the receive transducer element array include a greater number of transducer elements than a transmission transducer element array Tx. Further, the present embodiment is based on a configuration where the receive transducer element array for every transmission event includes all of the transducer elements 101a of the probe 101. Accordingly, for each transmission event, all the transducer elements 101a of the probe 101 are used for receiving ultrasound reflection in response to the transmission event, and a sequence of receive signals rf is generated for each transducer element 101a of the probe 101.

For each transmission event, the input unit 1091 generates a receive signal sub-frame data item rfl (a set of sequences of receive signals rf it has generated based on ultrasound reflection received in response to the transmission event), and outputs the receive signal sub-frame data item rfl to be stored in the data storage 114. Thus, the data storage 114 stores a sequence of receive signal sub-frame data items rfl.

### (2) Delay-and-Sum Calculator 1092

The delay-and-sum calculator 1092 generates an acoustic line signal sub-frame data item dsl for each transmission event. Specifically, the delay-and-sum calculator 1092 generates an acoustic line signal sub-frame data item dsl for a transmission event by generating an acoustic line signal for each measurement point Pij in the target area Bx for the transmission event. The delay-and-sum calculator 1092 generates an acoustic line signal for a measurement point Pij by performing delay processing on receive signals rf corresponding to the measurement point Pij that have been generated for a plurality of reception transducer elements Rpk, which are included in the receive transducer element array, and by summing the delayed receive signals rf. For each transmission event, the delay-and-sum calculator 1092 selects a reception transducer element array Rx based on the reception control signal from the reception condition setter 108. As already described above, the reception transducer element array Rx includes some of the transducer elements 101a of the probe 101, and the transducer elements 101a included in the reception transducer element array Rx are each referred to as a reception transducer element Rpk.

The delay-and-sum calculator 1092 includes a delay processor 10921 performing delay processing on receive signals rf, and a sum calculator 10922.

### (2-1) Delay Processor 10921

The delay processor 10921 is a circuit that, for each reception transducer element Rpk included in a reception transducer element array Rx, specifies a receive signal rf based on ultrasound reflection from a measurement point Pij, from among the sequence of receive signals rf for the reception transducer element Rpk. Specifically, the delay processor 10921 performs the specification of receive signals rf for the measurement point Pij by performing compensation by using delay amounts for the reception transducer elements Rpk. The delay amounts indicate the difference among the reception transducer elements Rpk in terms of the amount of time required for ultrasound reflection from the measurement point Pij to reach the reception transducer elements Rpk. Specifically, the delay processor 10921 calculates a delay amount for each reception transducer element Rpk by dividing the distance between the measurement point Pij and the reception transducer element Rpk by ultrasound velocity. FIGS. 6A and 6B are schematics illustrating how the delay processor 10921 calculates ultrasound propagation paths. FIG. 6A illustrates an example where the measurement point Pij is deeper than the transmission focal point FP, and FIG. 6B illustrates an example where the measurement point Pij is shallower than the transmission focal point FP. Each of FIGS. 6A and 6B illustrates a propagation path of ultrasound that is transmitted from the transmission transducer element array Tx and is reflected at a measurement point Pij before reaching a reception transducer element Rpk. As described above, the measurement point Pij is included in the target area Bx, which is set inside the ultrasound main irradiation area Ax.

### a) Calculation of Transmission Time

For each transmission event, the delay processor 10921 calculates a transmission time for each measurement point Pij set in the target area Bx for the transmission event, by calculating a transmission path through which transmitted ultrasound travels in the examination subject to reach the measurement point Pij, and dividing the transmission path by ultrasound velocity. The delay processor 10921 performs the calculation of the transmission time by using the following information, which the delay processor 10921 acquires from the transmission condition setter 105 for each transmission event: (i) the information indicating the transmission transducer elements 101a for the transmission event; (ii) the information indicating the transmission focal point FP for the transmission event; and (iii) the information indicating the position of the target area Bx inside the ultrasound main irradiation area Ax.

First, description is provided based on the transmission path illustrated in FIG. 6A. In FIG. 6A, ultrasound transmitted from the transmission transducer element array Tx first travels along path 401 to reach the transmission focal point FP, where the wavefront of the transmitted ultrasound focuses. Then, the transmitted ultrasound travels along path 402 to reach the measurement point Pij, which is deeper than the transmission focal point FP. Thus, the transmission time for the transmission path illustrated in FIG. 6A is the total of the time required for the transmitted ultrasound to travel through path 401 and the time required for the transmitted ultrasound to travel through path 402. Specifically, the transmission time for the transmission path illustrated in FIG. 6A can be calculated, for example, by dividing the total of the lengths of the paths 401 and 402 by ultrasound velocity inside the examination subject.

Next, description is provided based on the transmission path illustrated in FIG. 6B. In FIG. 6B, ultrasound transmitted from the transmission transducer element array Tx travels along path 404 to reach the measurement point Pij, which is shallower than the transmission focal point FP. The transmission time for the transmission path illustrated in FIG. 6B, where the measurement point Pij is shallower than the transmission focal point FP, is calculated assuming that the time required for the transmitted ultrasound to first travel along path 404 to reach the measurement point Pij and then travel along path 402 from the measurement point Pij to reach the transmission focal point FP is equal to the time required for the transmitted ultrasound to reach the transmission focal point FP by traveling along path 401. Thus, the transmission time required for transmitted ultrasound to travel through path 404 is the difference when subtracting the time required for the transmitted ultrasound to travel through path 402 from the time required for the transmitted ultrasound to travel through path 401. Specifically, the transmission time for the transmission path illustrated in FIG. 6B can be calculated, for example, by dividing the difference calculated by subtracting the length of path 402 from the length of path 401 by ultrasound velocity inside the examination subject.

The transmission condition setter 105 has set thereto preset values for the transmission focal point F. Thus, the length of the path 402 from the transmission focal point F to the measurement point Pij can be calculated geometrically.

### b) Calculation of Reception Time

Further, the delay processor 10921 calculates a reception time for each reception transducer element Rpk in the reception transducer element array Rx, by calculating a reception path through which transmitted ultrasound travels after being reflected at a measurement point P to reach the reception transducer element Rpk, and dividing the reception path by ultrasound velocity. The delay processor 10921 performs the calculation of the reception time by using the following information, which the delay processor 10921 acquires from the data storage 114 for each transmission event: information indicating the position of the reception transducer element array Rx for the transmission event.

Specifically, the delay processor 10921 performs the calculation of reception time for each reception transducer element Rpk supposing that ultrasound reflection is generated at the measurement point Pij due to a change in acoustic impedance at the measurement point Pij, and thus, based on a reception path where ultrasound reflection from the measurement point Pij travels along path 403 to return to the reception transducer element Rpk.

Since information indicating the positions of the reception transducer elements Rpk of the reception transducer element array Rx is acquired from the controller 115, the length of the path 403 from the measurement point Pij to each reception transducer element Rpk can be calculated geometrically.

### c) Calculation of Delay Amounts

Further, the delay processor 10921 calculates a total propagation time for each reception transducer element Rpk, based on the transmission time and the reception time for the reception transducer element Rpk. Further, the delay processor 10921, based on the total propagation time, calculates a delay amount to be applied to the sequence of receive signals rf for the reception transducer element Rpk. Specifically, for each reception transducer element Rpk, the delay processor 10921 calculates a total propagation time, which is the amount of time required for transmitted ultrasound to reach the reception transducer element Rpk after being reflected at the measurement point Pij. Further, based on the differences between the total propagation times for the reception transducer elements Rpk, the delay processor 10921 calculates, for each of the reception transducer elements Rpk, a delay amount to be applied to the sequence of receive signals rf for the reception transducer element Rpk.

### d) Delay Processing

FIGS. 7A and 7B are schematics illustrating the generation of an acoustic line signal for a measurement point Pij, performed by the delay-and-sum calculator 1092. FIG. 7A illustrates an example where the measurement point Pij is deeper than the transmission focal point FP, and FIG. 7B illustrates an example where the measurement point Pij is shallower than the transmission focal point FP. For each reception transducer element Rpk in the reception transducer element array Rx, the delay processor 10921 specifies a receive signal rf corresponding to the delay amount for the reception transducer element Rpk from among the sequence of receive signals rf for the reception transducer element Rpk. Further, the delay processor 10921 sets, for the reception transducer element Rpk, the specified receive signal rf as a receive signal rf based on ultrasound reflection from the measurement point Pij. For each transmission event, the delay processor 10921 performs the processing described above for each measurement point Pij included in the target area Bx for the transmission event by using sequences of receive signals rf acquired from the data storage 114 as input, as illustrated in FIGS. 7A and 7B.

### (2-2) Sum Calculator 10922

The sum calculator 10922 is a circuit that, for each measurement point Pij, generates a delayed-and-summed acoustic line signal for the measurement point Pij by using as input the receive signals rf for the reception transducer elements Rpk, which have been specified and output by the delay processor 10921, and summing the receive signals rf.

Alternatively, for each measurement point Pij, the sum calculator 10922 may generate a delayed-and-summed acoustic line signal for the measurement point Pij by multiplying the receive signals rf for the reception transducer elements Rpk by the weights for the reception transducer elements Rpk included in the reception apodization (sequence of weights), and summing the weighted receive signals rf. As already described above, a reception apodizaton for a reception transducer element array Rx is a numerical sequence of weight coefficients that are to be applied to receive signals rf for the respective reception transducer elements Rpk in the reception transducer element array Rx.

For each transmission event, the sum calculator 10922 performs the processing described above for each measurement point Pij in the target area Bx for the transmission event, as illustrated in FIGS. 7A and 7B, and thereby generates an acoustic line signal for each measurement point Pij in the target area Bx. For each transmission event, the sum calculator 10922 generates a plurality of acoustic line signals, each corresponding to a different one of the measurement points Pij in the target area Bx for the transmission event. The plurality of acoustic line signals generated in response to a single transmission event are collectively referred to as an acoustic line signal sub-frame data item dsl. Further, in the present disclosure, the term “sub-frame” is used to refer to a unit of signals that are generated in response to one transmission event and that correspond to a plurality of measurement points Pij included in a target area Bx.

The sum calculator 10922 sums the receive signals rf for the reception transducer elements Rpk, after the phases of the receive signals rf have been adjusted by the delay processor 10921. Due to this, the sum calculator 10922 is capable of increasing signal S/N ratio by combining receive signals rf based on ultrasound reflection from the same measurement point Pij, generated for different reception transducer elements Rpk. Thus, the sum calculator 10922 is capable of extracting a reception signal based on ultrasound reflection from the measurement point Pij.

### (3) Synthesizer 1093

As described above, transmission events are repeatedly performed while gradually shifting the transmission transducer element array Tx in the array direction each time, such that consequently, all transducer elements 101a of the probe 101 perform ultrasound transmission. The synthesizer 1093 is a circuit that generates a combined acoustic line signal frame data item ds by combining a plurality of acoustic line signal sub-frame data items dsl generated for transmission events. Further, the synthesizer 1093 repeatedly performs the generation of the combined acoustic line signal frame data item ds, and thereby generates a sequence of combined acoustic line signal frame data items ds. As illustrated in FIG. 4, the synthesizer 1093 includes a sub-frame combiner 10931 and an amplifier 10932.

### (3-1) Sub-Frame Combiner 10931

The sub-frame combiner 10931 reads out the acoustic line signal sub-frame data items dsl stored in the data storage 114. Further, the sub-frame combiner 10931 generates a combined acoustic line signal frame data item ds by combining the acoustic line signal sub-frame data items dsl. The combining of the acoustic line signal sub-frame data items dsl is performed according to the positions of measurement points Pij from which the acoustic line signals included in the acoustic line signal sub-frame data items dsl have been generated, such that in the process, a combined acoustic line signal is generated for each of the measurement points Pij. Specifically, the sub-frame combiner 10931 generates a combined acoustic line signal for each measurement point Pij by combining acoustic line signals corresponding to the measurement point Pij that are included in different acoustic line signal sub-frame data items dsl.

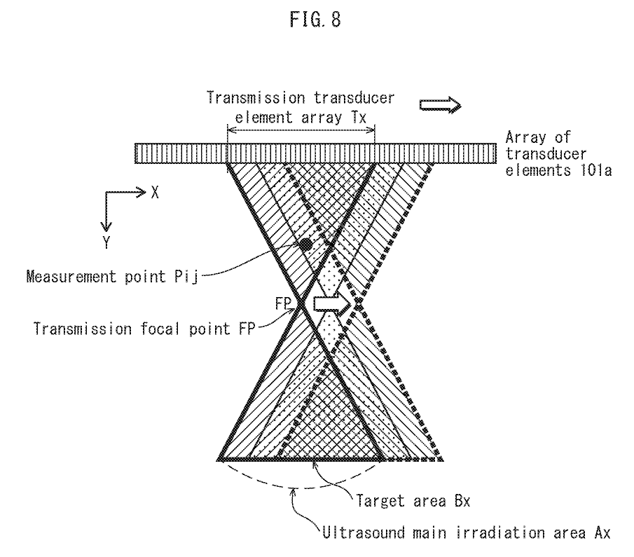

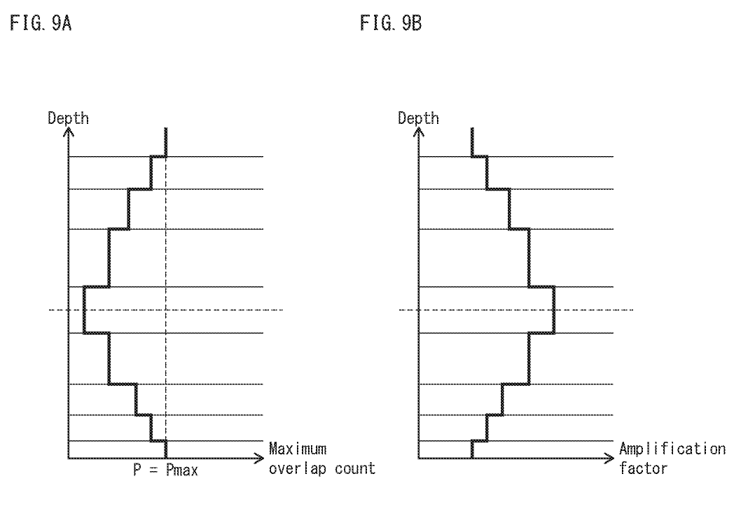

FIG. 8 is a schematic illustrating the combining of acoustic line signal sub-frame data items dsl, performed by the sub-frame combiner 10931. As already described above, transmission events are repeatedly performed while gradually shifting the transmission transducer element array Tx in the array direction each time. Due to this, the position of the target area Bx, which is set within the ultrasound main irradiation area Ax of each transmission event, also shifts in the array direction from one transmission event to another. In view of this, the sub-frame combiner 10931 generates a combined acoustic line signal frame data item ds covering target areas Bx for all transmission events, by combining acoustic line signal sub-frame data items dsl based on the positions of the measurement points Pij from which the acoustic lines signals included in the acoustic line signal sub-frame data items dsl have been generated.

Further, the sub-frame combiner 10931 generates combined acoustic line signals for the measurement points such that for a measurement point Pij that is included in multiple target areas Bx, values of acoustic line signals in different acoustic line signal sub-frame data items dsl for the same measurement point Pij are summed. Thus, some combined acoustic line signals may have a great value, depending upon the number of target areas Bx in which the corresponding measurement point Pij is included. In the following, the number of different target areas Bx in which a given measurement point Pij is included is referred to as an overlap count of the measurement point Pij, and the maximum value of the overlap count in the array direction is referred to as a maximum overlap count.

FIGS. 9A and 9B are schematics illustrating amplification performed by the synthesizer 1093. FIG. 9A illustrates a depth-direction variance among maximum overlap counts of combined acoustic line signals. Since the target areas Bx are hourglass-shaped areas, variance in the depth direction is observed among maximum overlap counts. Further, the intensity value of each combined acoustic line signal is dependent upon the maximum overlap count of the corresponding measurement point. Consequently, a variance in the depth direction is also observed among intensity values of combined acoustic line signals.

The sub-frame combiner 10931 outputs the combined acoustic line signal frame data item ds to the amplifier 10932.

### (3-2) Amplifier 10932

As already described above, a depth-direction variance is observed among the intensity values of combined acoustic line signals. In order to compensate for this depth-direction variance, the amplifier 10932 performs amplification by multiplying the combined acoustic line signals by amplification factors. The amplifier 10932 determines an amplification factor for a given combined acoustic line signal according to the number of acoustic line signals combined to yield the combined acoustic line signal, for generating the combined acoustic line signal frame data ds.

FIG. 9B illustrates a depth-direction variance among amplification factors used in the amplification. As described above, a depth-direction variance is observed among maximum overlap counts. Thus, to compensate for this variation among maximum overlap counts, the amplifier 10932 multiplies the combined acoustic line signals by respective amplification factors that are determined based on the maximum overlap counts and vary in the depth direction. The amplification with these amplification factors eliminates the depth-direction variance among values of combined acoustic line signals, which derives from the depth-direction variance among overlap counts. Thus, the amplified combined acoustic line signals have values that are averaged out somewhat in the depth direction. Here, a combined acoustic line signal frame data item ds is a group of combined acoustic line signals amplified in such a manner, each generated from a different measurement point Pij. Note that the present disclosure intends no limitation regarding how the above-described amplification factors are determined.

### 2.3 In-Puncture Reception Transducer Element Array Selector 113

The in-puncture reception transducer element array selector 113 is a circuit that selects, as a reception transducer element array Rx, a group of transducer elements 101a whose receive signals based on ultrasound reflection from the puncture needle 101b have relatively strong intensity, among the transducer elements 101a of the probe 101. The in-puncture reception transducer element array selector 113 performs the selection of this group of transducer elements 101a based on one or more receive signal sub-frame data items rfl. Here, the group of transducer elements 101a selected by the in-puncture reception transducer element array selector 113 is a group of transducer elements 101a whose receive signals based on ultrasound reflection from the puncture needle actually have or are expected to have stronger intensity than transducer elements 101a not selected by the in-puncture reception transducer element array selector 113.

Specifically, in the present embodiment, the in-puncture reception transducer element array selector 113 detects, from among sequences of receive signals rf for a plurality of transducer elements 101a, a sequence of receive signals rf including a receive signal with high intensity, and specifies a transducer element 101a corresponding to the detected sequence of receive signals rf as a high intensity receive transducer element 101a. Further, the in-puncture reception transducer element array selector 113 selects a group of transducer elements 101a including the specified high intensity receive transducer element 101a as a reception transducer element array Rx.

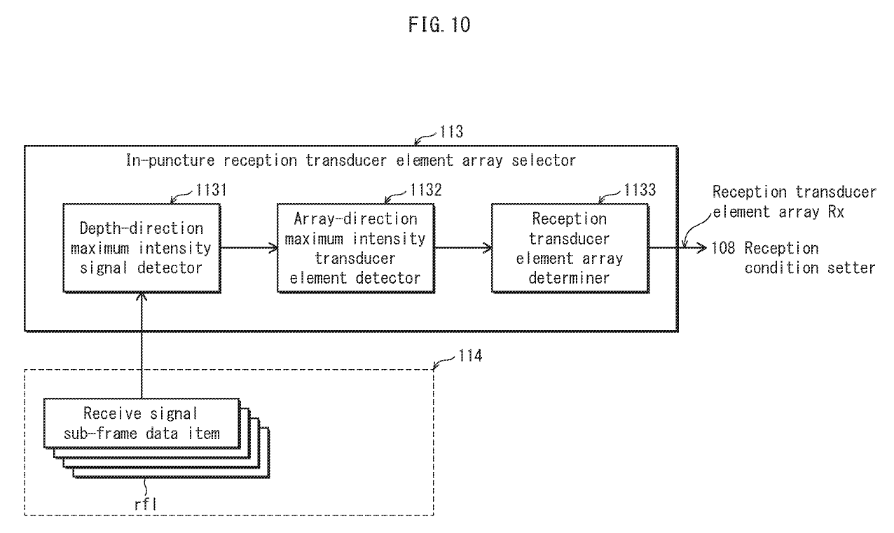

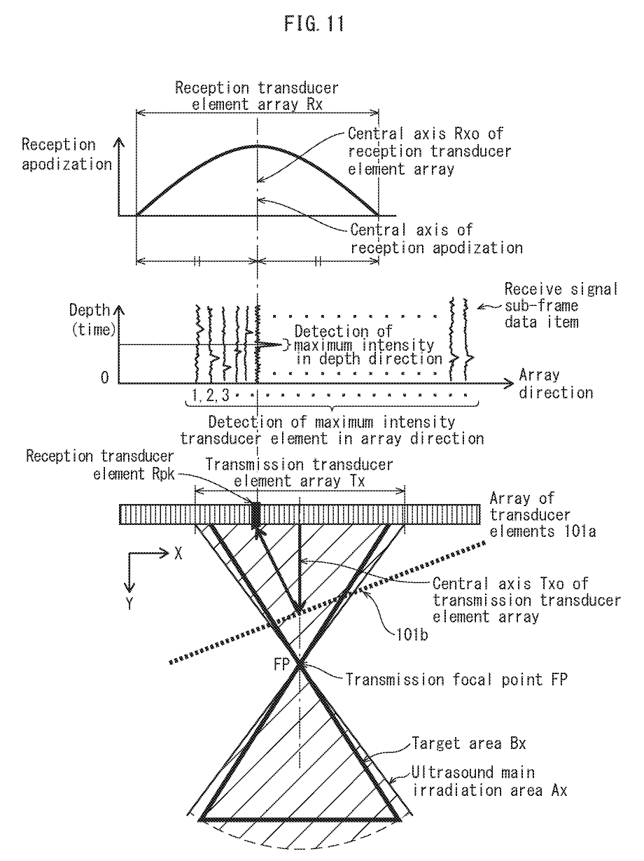

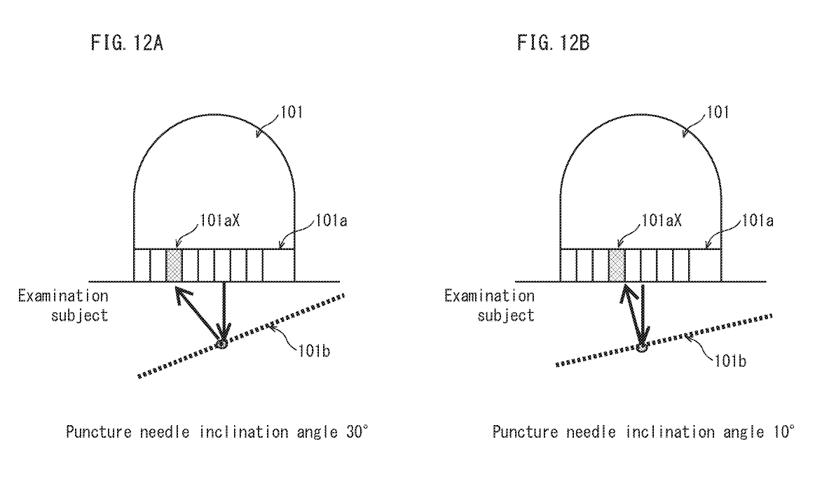

FIG. 10 illustrates functional blocks of the in-puncture reception transducer element array selector 113. Further, each of the following drawings is a schematic illustrating operations of the in-puncture reception transducer element array selector 113: FIG. 11, FIGS. 12A and 12B, FIGS. 13A through 13D, and FIGS. 14A through 14D. As illustrated in FIG. 10, the in-puncture reception transducer element array selector 113 includes: a depth-direction high-intensity signal detector 1131; an array-direction maximum intensity transducer element detector 1132; and a reception transducer element array determiner 1133.

(1) Depth-Direction High-Intensity Signal Detector 1131

The in-puncture reception transducer element array selector 113 reads out one or more receive signal sub-frame data items rfl from the data storage 114.

The depth-direction high-intensity signal detector 1131 performs analysis of the plurality of sequences of receive signals rf included in a receive signal sub-frame data item rfl. In this analysis, the depth-direction high-intensity signal detector 1131 determines whether or not each sequence of receive signals rf includes a signal portion (i.e., a receive signal rf) with an intensity higher than or equal to a predetermined threshold, to detect sequences of receive signals rf including receives signals rf with intensities higher than or equal to a predetermined threshold, as illustrated in the middle portion of FIG. 11. As already described above, the puncture needle 101b generates specular ultrasound reflection. Thus, signals based on ultrasound reflection from the puncture needle 101b have extremely higher intensity than signals based on ultrasound reflection from body tissues. The predetermined threshold is set to distinguish signals based on ultrasound reflection from the puncture needle 101b from signals based on ultrasound reflection from body tissues. Accordingly, a transducer element 101a having received ultrasound reflection from the puncture needle 101b can be detected by detecting a sequence of receive signals rf including a receive signal rf with an intensity higher than or equal to the predetermined threshold.

The depth-direction high-intensity signal detector 1131 performs the extraction, from sequences of receive signals rf, of receives signals rf with intensities higher than or equal to the predetermined threshold, for each receive signal sub-frame data item rfl that is read out.

### (2) Array-Direction Maximum Intensity Transducer Element Detector 1132

As illustrated in the middle portion of FIG. 11, the array-direction maximum intensity transducer element detector 1132 detects, from among sequences of receive signals rf detected as including receive signals rf with intensities higher than or equal to the predetermined threshold in a receive signal sub-frame data item rfl, a sequence of receives signals rf that includes the greatest or close-to-greatest receive signal rf among the receive signals rf with intensities higher than or equal to the predetermined threshold. Further, the array-direction maximum intensity transducer element detector 1132 specifies a transducer element 101a corresponding to the detected sequence of receive signals rf as a high intensity receive transducer element 101aX. By detecting a transducer element 101a corresponding to the greatest or close-to-greatest one among the receive signals rf with intensities higher than or equal to the predetermined threshold, a transducer element 101a whose receive signal rf based on ultrasound reflection from the puncture needle 101b has relatively high intensity (i.e., a high intensity receive transducer element 101aX) can be detected.

The array-direction maximum intensity transducer element detector 1132 performs the specification of a high intensity receive transducer element 101aX with respect to each receive signal sub-frame data item rfl that is read out.

### (3) Reception Transducer Element Array Determiner 1133

As illustrated in the upper portion of FIG. 11, the reception transducer element array determiner 1133 is, for example, capable of selecting, as a reception transducer element array Rx, a group of transducer elements 101a including, at an array-direction center position thereof, a high intensity receive transducer element 101aX acquired from one receive signal sub-frame data item rfl. When delay-and-sum processing is performed by using a reception apodization, it is preferable that the central axis of the reception apodization in the array direction be set to the high intensity receive transducer element 101aX.

With this configuration, a high intensity receive transducer element 101aX can be detected and a group of transducer elements including the high intensity receive transducer element 101aX at the array-direction center position can be selected as a reception transducer element array Rx, even when the insertion angle (inclination angle) of the puncture needle 101b with respect to the examination subject changes as illustrated in FIGS. 12A and 12B.

Meanwhile, as already described above, due to transmitted ultrasound being focused ultrasound, the angle θ at which the two straight lines that define the boundary of the ultrasound main irradiation area Ax intersect at the transmission focal point FP is typically approximately greater than or equal to 14° and smaller than or equal to 28°. Due to this, when the transmitted ultrasound is focused ultrasound, an array-direction variance, within the angle θ, is observed in the angle between the puncture needle 101b and the transmitted ultrasound, as illustrated in FIGS. 13A through 13C. Due to this, a plurality of transducer elements 101a with greatest or close-to-greatest signal intensities may be detected from one receive signal sub-frame data item rfl acquired in response to one transmission event. That is, a plurality of high intensity receive transducer elements 101aX may be detected from one receive signal sub-frame data item rfl. In such a case, the reception transducer element array determiner 1133 may select, as a reception transducer element array Rx, a group of transducer elements 101a including all of these high intensity receive transducer elements 101aX, as illustrated in FIG. 13D. In this case as well, when delay-and-sum processing is performed by using a reception apodization, it is preferable that the central axis of the reception apodization in the array direction be set to the center one of the high intensity receive transducer elements 101aX in the array direction. This configuration enables selecting, as a reception transducer element array Rx, a group of transducer elements 101a including a plurality of high intensity receive transducer elements 101aX.

Alternatively, the reception transducer element array determiner 1133 may select, as a reception transducer element array Rx, a group of transducer elements 101a including a plurality of high intensity receive transducer elements 101aX acquired from a plurality of receive signal sub-frame data items rfl. When the position of the transmission transducer element array Tx shifts in the array direction between transmission events, high intensity receive transducer elements 101aX detected from different receive signal sub-frame data items rfl, each corresponding to a different one of the transmission events, take different positions, as illustrated in FIGS. 14A through 14C. In this case, the reception transducer element array determiner 1133 may select, as a reception transducer element array Rx, a group of transducer elements 101a including the high intensity receive transducer elements 101aX detected from different receive signal sub-frame data items rfl and including the center one of the high intensity receive transducer elements 101aX in the array direction at the center thereof in the array direction, as illustrated in FIG. 14D. In this case as well, it is preferable that the central axis of the reception apodization in the array direction be set to the center one of the high intensity receive transducer elements 101aX in the array direction. This configuration enables selecting, as a reception transducer element array Rx, a group of transducer elements 101a whose receive signals rf based on ultrasound reflection from the puncture needle 101b have relatively high intensity across a plurality of transmission events.

The reception transducer element array determiner 1133 outputs information indicating the position of the reception transducer element array Rx it has selected to the reception condition setter 108. Meanwhile, when not detecting a group of transducer elements 101a whose receive signals based on ultrasound reflection from the puncture needle 101b have relatively high intensity, the in-puncture reception transducer element array selector 113 does not output any information indicating a reception transducer element array Rx to the reception condition setter 108.

### 3. Other Components

The B-mode image generator 112 acquires a sequence of combined acoustic line signal frame data items ds from the data storage 114 as input. The B-mode image generator 112 converts combined acoustic line signals in each combined acoustic line signal frame data item ds into luminance signals representing the intensities of the combined acoustic line signals by performing processing such as envelope detection and logarithmic compression, and further converts the luminance signals into coordinates on an orthogonal coordinate system. Thus, the B-mode image generator 112 generates a sequence of B-mode image frame data items. Here, the B-mode image generator 112 may use any conventional method to generate a sequence of B-mode image frame data items from a sequence of combined acoustic line signal frame data items. Further, the B-mode image generator 112 outputs the sequence of B-mode image frame data items to the data storage 114 to be stored therein. The display controller 116 forms display images from the B-mode image frame data items, and causes the display unit 103 to display the display images. The display controller 116 may be further configured to perform processing such as emphasizing or coloring of pixels of the B-mode images corresponding to the puncture needle 101b, and to output the processed pixels to the display unit 103.

The data storage 114 is a recording medium capable of storing sequences of receive signals rf, acoustic line signal sub-frame data items dsl, combined acoustic line signal frame data items ds, B-mode image frame data items, etc., in the order they are received.

The controller 115 controls each of the functional blocks of the ultrasound diagnostic device 100 based on instructions from the operation receiver 102. The controller 115 may be implemented, for example, by using a processor such as a CPU.

### <Operations>

### 1. Beam Forming for Generating Acoustic Line Signal Sub-Frame Data Items

The following describes beam forming for the generation of acoustic line signal sub-frame data items dsl, performed by the ultrasound diagnostic device 100, whose structure has been described up to this point.

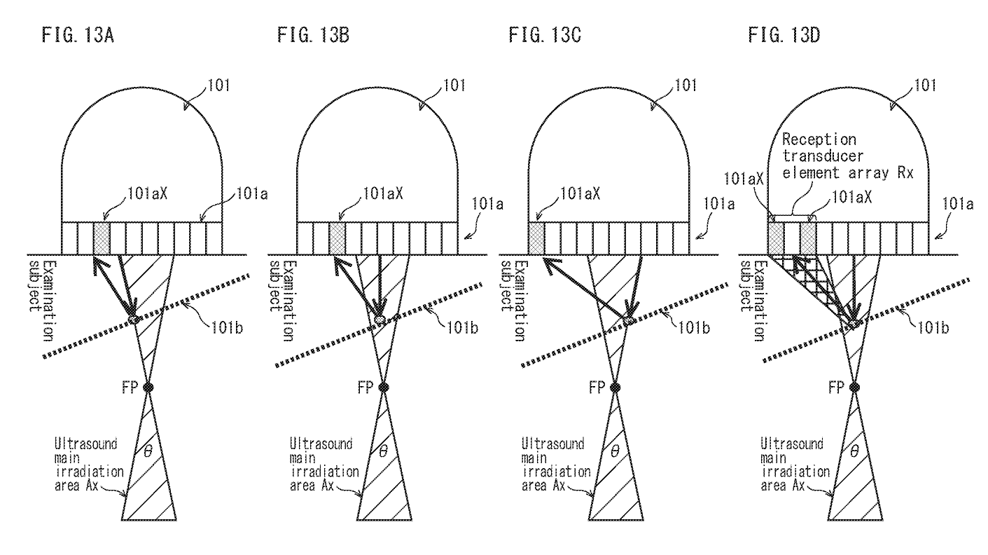

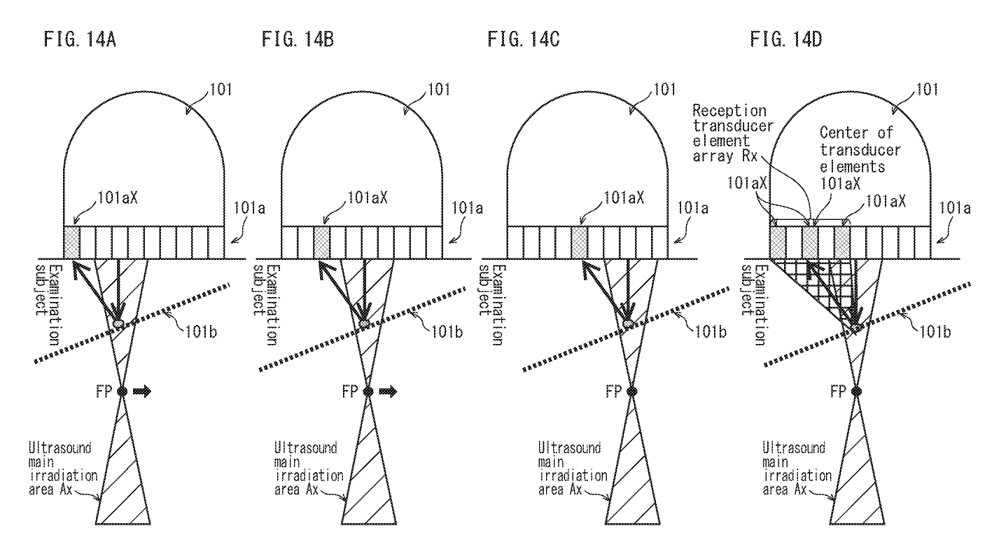

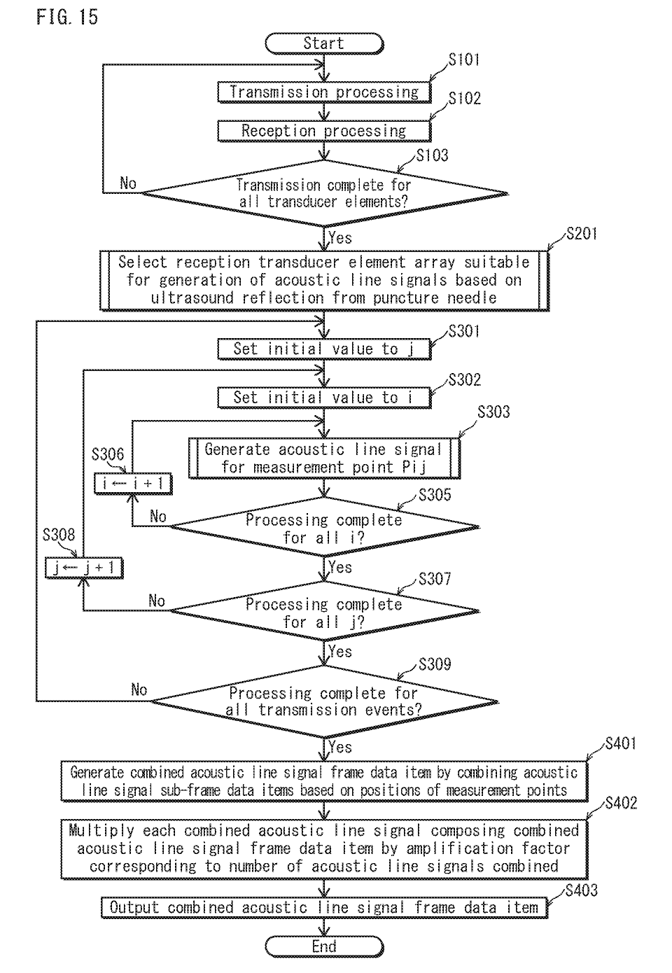

FIG. 15 is a flowchart illustrating beam forming by the ultrasound diagnostic device 100.

First, in Step S101, the transmission beam former 106 performs transmission processing (a transmission event) of supplying a transmission signal triggering transmission of an ultrasound beam to each transmission transducer element 101a of the transmission transducer element array Tx set by the transmission condition setter 105.

Next, in Step S102, the reception beam former 109 generates receive signals rf based on electric signals yielded through the reception of ultrasound reflection by the probe 101, and outputs the receive signals rf to be stored in the data storage 114. Then, a determination is made of whether or not a predetermined number of transmission events have been performed (Step S103). When the predetermined number of transmission events have not been performed yet, processing returns to S101, and another transmission event is performed after shifting the transmission transducer element array Tx by a predetermined pitch in the array direction. Meanwhile, when the predetermined number of transmission events have been performed, processing proceeds to Step S201.

Subsequently in Step S201, the in-puncture reception transducer element array selector 113 selects a reception transducer element array Rx based on one or more receive signal sub-frame data items rfl, and outputs the information indicating the result of the selection to the reception condition setter 108. Here, the in-puncture reception transducer element array selector 113 selects, as a reception transducer element array Rx, a group of transducer elements 101a whose receive signals based on ultrasound reflection from the puncture needle 101b have relatively high intensity, or that is, a group of transducer elements 101a suitable for generation of acoustic line signals from the puncture needle 101b. The processing in Step S201 is described in detail later in the present disclosure.

Subsequently, coordinate values i and j indicating a position of a measurement point Pij that is included in the target area Bx set by the reception condition setter 108 are initialized (set to the respective minimum possible values in the target area Bx) (Steps S301 and S302). Then, an acoustic line signal is generated for the current measurement point Pij (Step S303), based on the data of sequences of receive signals rf for the reception transducer elements Rpk in the reception transducer element array Rx set by the reception condition setter 108, which are included in a receive signal sub-frame data item rfl for the processing-target transmission event. The processing in Step S303 is described in detail later in the present disclosure.

Further, an acoustic line signal is generated for each measurement point Pij (each illustrated in FIGS. 7A and 7B as a black dot) that is included in the target area Bx by repeating Step S303 while incrementing the coordinate values i and j. Subsequently, a determination is made of whether an acoustic line signal has been generated for every measurement point Pij included in the target area Bx (Steps S305, S307). When an acoustic line signal has not yet been generated for every measurement point Pij within the target area Bx, the coordinate values i and j are incremented (Steps S306 and S308), yielding an acoustic line signal for another measurement point Pij (Step S303). Meanwhile, when an acoustic line signal has already been generated for every measurement point Pij within the target area Bx, processing proceeds to Step S309. At this point, an acoustic line signal has already been generated for each measurement point Pij that is included in the target area Bx set inside the ultrasound main irradiation area Ax for one transmission event, and the acoustic line signals have been output to and stored to the data storage 114. In other words, an acoustic line signal sub-frame data item dsl for one transmission event has been generated, and has been output to and stored to the data storage 114.

Subsequently, a determination is made of whether or not the generation of acoustic line signals has been performed for every transmission event having been performed (Step S309). When the generation of acoustic line signals has not yet been performed for every transmission event having been performed, processing returns to Step S301, and an acoustic line signal sub-frame data item dsl is generated based on ultrasound transmitted in a subsequent transmission event (Steps S301 through S307). Meanwhile, when the generation of acoustic line signals has been performed for every transmission event having been performed, processing is terminated.

Subsequently, in Step S401, the sub-frame combiner 10931 generates a combined acoustic line signal frame data item ds (Step S401). Specifically, the sub-frame combiner 10931 reads out acoustic line signal sub-frame data items dsl for all sub-frames from the data storage 114, and combines the acoustic line signal sub-frame data items dsl according to the positions of the measurement points Pij, such that in the process, a combined acoustic line signal is generated for each measurement point Pij. Thus, a combined acoustic line signal frame data item ds is generated. Subsequently, the amplifier 10932 multiplies each combined acoustic line signal included in the combined acoustic line signal frame data item ds by an amplification factor determined based on the number of acoustic line signals combined to yield the combined acoustic line signal (Step S402). Then, the amplifier 10932 outputs the amplified combined acoustic line signal frame data item ds to the data storage 114 (Step S403), and processing is terminated.

Thus, the generation of a combined acoustic line signal frame data item ds is completed.

Note that a sequence of combined acoustic line signal frame data items ds is generated by the generation of the combined acoustic line signal frame data item ds illustrated in FIG. 15 being performed repeatedly. The reception beam former 109 outputs the sequence of combined acoustic line signal frame data items ds to be stored in the data storage 114.

### 2. Details of Processing in Step S201

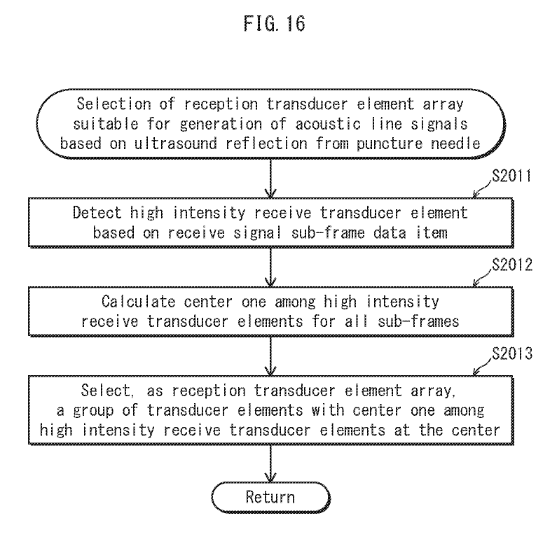

The following describes the processing in Step S201 of selecting a reception transducer element array Rx suitable for generation of acoustic line signals from the puncture needle 101b. FIG. 16 is a flowchart illustrating processing by the in-puncture reception transducer element array selector 113 of selecting such a reception transducer element array Rx.

The in-puncture reception transducer element array selector 113 reads out a plurality of receive signal sub-frame data items rfl from the data storage 114.

In Step S2011, the depth-direction high-intensity signal detector 1131 analyzes the sequences of receive signals rf included in each receive signal sub-frame data item rfl, and extracts, from the sequences of receive signals rf, receive signals rf having relatively high intensities (i.e., intensities higher than or equal to the predetermined threshold). Here, transducer elements 101a having received ultrasound reflection from the puncture needle 101b can be detected by detecting sequences of receive signals rf including receive signals rf with intensities higher than or equal to the predetermined threshold. This is since the puncture needle 101b generates specular ultrasound reflection.

Further, the array-direction maximum intensity transducer element detector 1132 detects, from among receive signals rf having intensities higher than or equal to the predetermined threshold having been detected in each receive signal sub-frame data item rfl, a receive signal rf having the greatest or close-to-greatest intensity. Then, the array-direction maximum intensity transducer element detector 1132 sets the transducer element 101a having received a sequence of receive signals rf including such a receive signal rf as a high intensity receive transducer element 101aX (i.e., specifies the transducer element 101a as a transducer element 101a whose receive signal rf based on ultrasound reflection from the puncture needle 101b has relatively high signal intensity). The array-direction maximum intensity transducer element detector 1132 performs this detection for each receive signal sub-frame data item rfl that is read out, and specifies a high intensity receive transducer element 101aX in each receive signal sub-frame data item rfl that is read out.

In Step S2012, the reception transducer element array determiner 1133 specifies the center one of the high intensity receive transducer elements 101aX in the array direction, each specified in a different receive signal sub-frame data item rfl.

In Step S2013, the reception transducer element array determiner 1133 selects, as a reception transducer element array Rx, a group of transducer elements 101a including the center one of the high intensity receive transducer elements 101aX in the array direction at the center thereof in the array direction. Here, it is preferable that the reception transducer element array Rx be selected to include all the high intensity receive transducer elements 101aX, each specified in a different receive signal sub-frame data item rfl. This configuration enables selecting, as a reception transducer element array Rx, a group of transducer elements 101a whose receive signals rf based on ultrasound reflection from the puncture needle 101b have relatively high intensity across a plurality of transmission events.

The reception transducer element array determiner 1133 outputs information indicating the position of the reception transducer element array Rx it has selected to the reception condition setter 108.

### 3. Details of Processing in Step S303

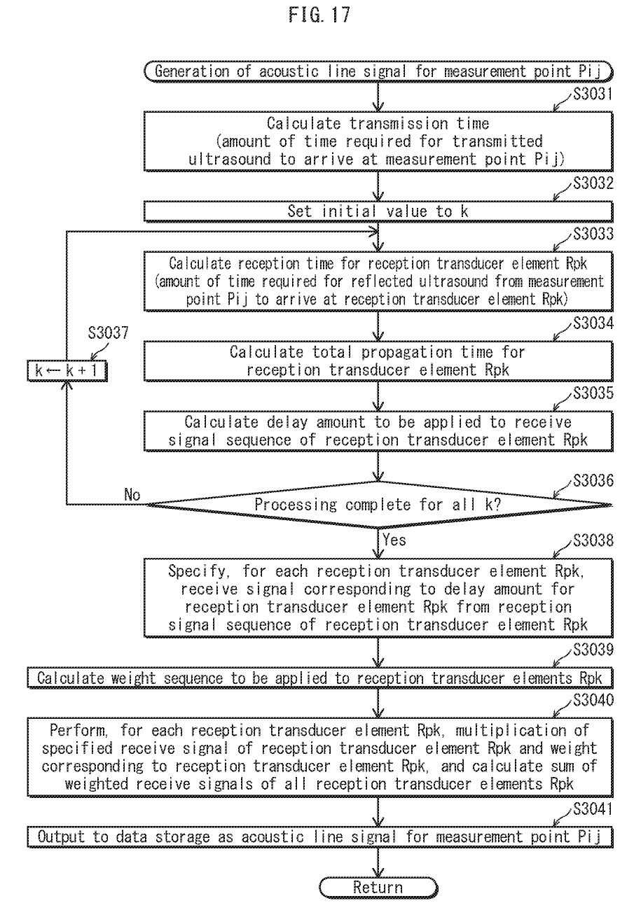

The following describes the processing in Step S303 of generating an acoustic line signal for a measurement point Pij. FIG. 17 is a flowchart illustrating the processing of generating an acoustic line signal for a measurement point Pij, performed by the delay-and-sum calculator 1092.

The delay-and-sum calculator 1092 receives, from the reception condition setter 108, information indicating a target area Bx, information indicating a reception transducer element array Rx, and information indicating a reception apodization.

First, in Step S3031, the delay processor 10921 calculates, for the current measurement point Pij in the target area Bx, a transmission time required for transmitted ultrasound to arrive at the current measurement point Pij.

As described above, when the measurement point Pij is shallower than the transmission focal point FP, the transmission time is calculated by calculating a transmission path 404 from the transmission transducer element array Tx to the measurement point Pij, and dividing the length of the transmission path 404 by ultrasound velocity cs. Specifically, the transmission path is calculated by calculating the difference (401-402) between a path 401 from the center position of the transmission transducer element array Tx in the array direction to the transmission focal point FP and a path 402 from the transmission focal point FP to the measurement point Pij.

Meanwhile, as also already described above, when the measurement point Pij is deeper than the transmission focal point FP, the transmission time is also calculated by calculating a transmission path from the transmission transducer element array Tx to the measurement point Pij, and dividing the length of this transmission path by ultrasound velocity cs, but this time, the transmission path is calculated by calculating the sum (401+402) of the path 401 from the center position of the transmission transducer element array Tx in the array direction to the transmission focal point FP and the path 402 from the transmission focal point FP to the measurement point Pij

Subsequently, value k, which identifies a target reception transducer element Rpk in the reception transducer element array Rx, is initialized (set to the minimum possible value in the reception transducer element array Rx) (Step S3032). Then, a reception time for the target reception transducer element Rpk is calculated (Step S3033). The reception time is the amount of time required for ultrasound reflection from the measurement point Pij, originating from the transmitted ultrasound, to arrive at the reception transducer element Rpk (Step S3033).

The reception time for the target reception transducer element Rpk can be calculated by dividing, by ultrasound velocity cs, the geometrically-calculable length of path 403 from the current measurement point Pij to the target reception transducer element Rk. Further, from a sum of the transmission time and the reception time for the target reception transducer element Rpk, the total propagation time required for ultrasound transmitted from the transmission transducer element array Tx to arrive at the target reception transducer element Rpk after being reflected at the current measurement point Pij is calculated (Step S3034). Further, based on the difference in total propagation time among the reception transducer elements Rpk of the reception transducer element array Rx, a delay amount for the target reception transducer element Rpk is calculated (Step S3035).

Subsequently, a determination is made of whether or not a delay amount has been calculated for every reception transducer element Rpk in the reception transducer element array Rx (Step S3036). When a delay amount has not yet been calculated for one or more of the reception transducer elements Rpk, the value k is incremented (Step S3037), and a delay amount for another reception transducer element Rpk is calculated (Step S3035). Meanwhile, when a delay amount has been calculated for every reception transducer element Rpk in the reception transducer element array Rx, processing proceeds to Step S3038. Note that at this point, a delay amount has already been calculated for each reception transducer element Rpk in the reception transducer element array Rx. The delay amount for a given reception transducer element Rpk indicates the delay with which ultrasound reflection from the current measurement point Pij reaches the reception transducer element Rpk.

In Step S3038, the delay processor 10921, for each reception transducer element Rpk, specifies a receive signal rf based on ultrasound reflection from the current measurement point Pij. Specifically, the delay processor 10921 reads, from the data storage 114, sequences of receive signals rf corresponding to the reception transducer elements Rpk in the reception transducer element Rx included in a receive signal sub-frame data item rfl. Further, for each reception transducer element Rpk, the delay processor 10921 specifies a receive signal rf corresponding to a time point after subtraction of the delay amount for the reception transducer element Rpk in the corresponding sequence of receive signals rf, as a receive signal based on ultrasound reflection from the current measurement point Pij.

Subsequently, an undepicted weight calculator calculates the reception apodization (numerical sequence of weight coefficients) for the reception transducer element array Rx, so that the maximum weight is set with respect to the reception transducer element Rpk located at the center position of the reception transducer element array Rx in the array direction (Step S3039). Subsequently, the sum calculator 10922 generates an acoustic line signal for the current measurement point Pij by first multiplying the specified receive signals rf for the respective reception transducer element Rpk by the corresponding weights in the reception apodization, and summing the weighted receive signals rf (Step S3040). The acoustic line signal for the current measurement point Pij so generated is output to the data storage 114 to be stored therein (Step S3041).

This completes the processing in Step S303 in FIG. 15.

### <Effects>

As described up to this point, the ultrasound diagnostic device 100 pertaining to embodiment 1 is an ultrasound diagnostic device to which an ultrasound probe 101 having a plurality of transducer elements 101 arrayed along an array direction is connectable, wherein the ultrasound diagnostic device 100: (i) repeatedly performs transmission events, each by selecting a first group (transmission transducer element array) Tx of transducer elements 101a from among the plurality of transducer elements 101a and causing the first group Tx to transmit ultrasound towards an examination subject having a puncture needle 101b inserted therein; (ii) for each of the transmission events, generates a sub-frame data item dsl based on ultrasound reflection received in response to the transmission event, the sub-frame data item dsl including acoustic line signals; and (iii) combines sub-frame data items dsl for the transmission events to generate a frame data item ds, the frame data item ds including combined acoustic line signals each being an aggregate of ones of the acoustic line signals, and the ultrasound diagnostic device 100 includes an ultrasound signal processing circuit 150 operating as: a transmitter 107 that performs each of the transmission events by selecting the first group Tx and causing the first group Tx to transmit ultrasound focusing inside the examination subject, the transmitter 107 gradually shifting the first group Tx in the array direction between transmission events; and a receiver 110 that: (i) for each of the transmission events, selects at least some of the plurality of transducer elements 101a and generates a sequence of receive signals rf for each of the selected transducer elements 101a based on ultrasound reflection that the selected transducer element 101a has received from the examination subject in response to the transmission event; (ii) selects a second group (reception transducer element array) Rx of transducer elements 101a, the transducer elements 101a in the second group Rx being ones among the plurality of transducer elements 101a whose receive signals rf based on ultrasound reflection from the puncture needle 101b have high intensity; (iii) for each of the transmission events, sets a target area Bx for generating the sub-frame data item dsl inside a virtual area of the examination subject that receives ultrasound transmitted in the transmission event, and generates the sub-frame data item dsl by performing, for each of a plurality of measurement points Pij in the target area Bx, delay-and-sum processing on receive signals rf, for the transducer elements 101a in the second group Rx, based on ultrasound reflection from the measurement point Pij; and (iv) generates the frame data item ds by combining the sub-frame data items dsl for the transmission events.

With this structure, the ultrasound diagnostic device 100 is capable of producing ultrasound images imaging a puncture needle with high visible perceptibility, with reception beam forming utilizing the synthetic aperture method. Specifically, the ultrasound diagnostic device 100 utilizes the synthetic aperture method and repeatedly performs transmission events while gradually shifting the transmission transducer element array Tx in the array direction. Further, the ultrasound diagnostic device 100 performs delay-and-sum processing for each transmission event by using a reception transducer element array Rx composed of reception transducer elements Rpk whose receive signals rf based on ultrasound reflection from the puncture needle have high intensity.

When performing ultrasound examination by using the ultrasound diagnostic device 100 with the puncture needle 101b inserted in the examination subject, signals based on ultrasound reflection from the puncture needle 101b have extremely higher intensity than signals based on ultrasound reflection from body tissues. This is since the puncture needle 101b produces specular ultrasound reflection and signals based on specular reflection have high directivity, whereas body tissues mainly produce diffuse ultrasound reflection. The ultrasound diagnostic device 100 distinguishes signals based on ultrasound reflection from the puncture needle 101b from signals based on ultrasound reflection from body tissues by using a predetermined threshold. Specifically, the ultrasound diagnostic device 100 detects a transducer element 101a whose receive signal rf has intensity higher than or equal to the predetermined threshold, specifies such a transducer element 101a as a transducer element 101a having received ultrasound reflection from the puncture needle 101b, and selects a group of transducer elements 101a including such a transducer element 101a as a reception transducer element array Rx. Thus, the ultrasound diagnostic device 100 is capable of selecting, as a reception transducer element array Rx, a group of transducer elements 101a whose receive signals rf based on ultrasound reflection from the puncture needle 101b have relatively high intensity. The ultrasound diagnostic device 100 is capable of producing ultrasound images imaging the puncture needle 101b with high visual perceptibility by performing, for each measurement point Pij in a target area Bx, delay-and-sum processing on receive signals rf corresponding to the reception transducer elements 101a of a reception transducer element array Rx selected in such a manner to generate an acoustic line signal sub-frame data item dsl for the target area Bx.

Meanwhile, the generation of acoustic line signals for the target area Bx, performed by using the selected reception transducer element array Rx, also allows generating acoustic line signals based on ultrasound reflection from body tissues. This is since, even when a group of transducer elements 101a whose receive signals rf based on ultrasound reflection from the puncture needle 101b have high intensity is selected as a reception transducer element array Rx, the transducer elements 101a included in the selected reception transducer element array Rx also have received ultrasound reflection from body tissues of the examination subject due to body tissues mainly producing diffuse ultrasound reflection.

Further, the ultrasound diagnostic device 100 is capable of selecting a reception transducer element array Rx through simple processing that does not require prior execution of delay-and-sum processing, which typically has great processing load. Specifically, in the ultrasound diagnostic device 100, the in-puncture reception transducer element array selector 113 detects, from among sequences of receive signals rf, a sequence of receive signals rf including a receive signal rf with high intensity, and specifies a transducer element 101a corresponding to the detected sequence of receive signals rf as a high intensity receive transducer element 101aX. Further, the in-puncture reception transducer element array selector 113 selects, as a reception transducer element array Rx, a group of transducer elements 101a including the specified high intensity receive transducer element 101aX. As such, the ultrasound diagnostic device 100 is capable of selecting, as a reception transducer element array Rx, a group of transducer elements 101a whose receive signals rf based on ultrasound reflection from the puncture needle 101b have high intensity with a simple circuit structure. Note that the method described in embodiment 1 is a non-limiting example of a method of selecting a reception transducer element array Rx based on receive signals. That is, the selection of a reception transducer element array Rx can be performed by using other methods as long as the selection of a reception transducer element array Rx is performed based on receive signal intensity.

Specifically, the in-puncture reception transducer element array selector 113 is configured to enable selection of a reception transducer element array Rx based on receive signals. In FIG. 10, the in-puncture reception transducer element array selector 113 is illustrated to include the depth-direction high intensity signal detector 1131 and the array-direction maximum intensity transducer element detector 1132. However, for example, the in-puncture reception transducer element array selector 113 need not have such a structure. That is, the in-puncture reception transducer element array selector 113 may have any structure as long as it is capable of selecting, based on receive signal intensity, a group of transducer elements 101a whose receive signals rf based on ultrasound reflection from the puncture needle 101b have high intensity as a reception transducer element array Rx. For example, the in-puncture reception transducer element array selector 113 may be configured to detect a receive signal rf having the greatest or close-to-greatest signal intensity and a high intensity receive transducer element 101aX corresponding to the detected receive signal rf from a receive signal sub-frame data item rfl through a single process, and to select a group of transducer elements 101a including the detected high intensity receive transducer element 101aX at the center thereof in the array direction as a reception transducer element array Rx. Alternatively, the in-puncture reception transducer element array selector 113 may be configured to detect receive signals rf having intensities higher than or equal to the predetermined threshold and transducer elements 101a corresponding to such receive signals rf from a receive signal sub-frame data item rfl, and to select a group of transducer elements 101a including these transducer elements 101a as a reception transducer element array Rx. Alternatively, the in-puncture reception transducer element array selector 113 may be configured to use other processing methods.

### Embodiment 2

The following describes an ultrasound diagnostic device 100A pertaining to embodiment 2. In the ultrasound diagnostic device 100 pertaining to embodiment 1, the in-puncture reception transducer element array selector 113 selects a group of transducer elements 101a whose receive signals rf based on ultrasound reflection from the puncture needle 101b have high intensity, as illustrated in FIGS. 10 and 11. Specifically, the in-puncture reception transducer element array selector 113 detects, from among sequences of receive signals rf corresponding to a plurality of transducer elements 101a, a sequence of receive signals rf including a high intensity receive signal rf, and specifies a transducer element 101a corresponding to the detected receive signal rf as a high intensity receive transducer element 101aX. Further, the in-puncture reception transducer element array selector 113 selects, as a reception transducer element array Rx, a group of transducer elements 101a including the high intensity receive transducer element 101aX. However, the selection of a reception transducer element array Rx need not be performed according to this specific method, and other methods may be used as long as a group of transducer elements 101a suitable for the generation of acoustic line signals from the puncture needle 101b is selected as a reception transducer element array Rx.

Specifically, the ultrasound diagnostic device 100A differs from the ultrasound diagnostic device 100 for including an in-puncture reception transducer element array selector that specifies the position/inclination angle at which the puncture needle has been imaged based on characteristics of acoustic line signals in one or more acoustic line signal sub-frame data items dsl, and performs the selection of a reception transducer element array Rx based on the specified position/inclination angle of the puncture needle.

### <Structure>

The following describes the structure of the ultrasound diagnostic device 100A.

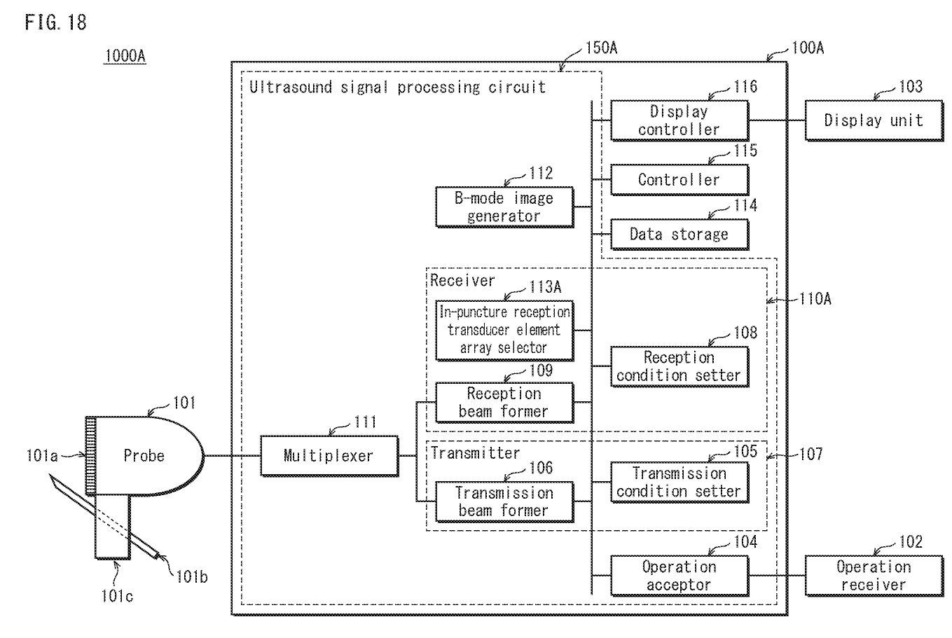

FIG. 18 illustrates functional blocks of an ultrasound diagnostic system 1000A including the ultrasound diagnostic device 100A. Since the ultrasound diagnostic device 100A differs from the ultrasound diagnostic device 100 for including an in-puncture reception transducer element array selector 113A, the following description focuses on the structure of the in-puncture reception transducer element array selector 113A. Since the rest of the components of the ultrasound diagnostic device 100A other than the in-puncture reception transducer element array selector 113A are similar to those in the ultrasound diagnostic device 100, description is not provided of the rest of the components of the ultrasound diagnostic device 100A.

The in-puncture reception transducer element array selector 113A is a circuit that selects a reception transducer element array Rx based on the position/inclination angle at which the puncture needle 101b has been imaged. Specifically, the in-puncture reception transducer element array selector 113A extracts, from an acoustic line signal sub-frame data item dsl, a linear area composed of acoustic line signals with high intensity. Further, the in-puncture reception transducer element array selector 113A, based on the linear area, specifies the position/inclination angle at which the puncture needle 101b has been imaged.

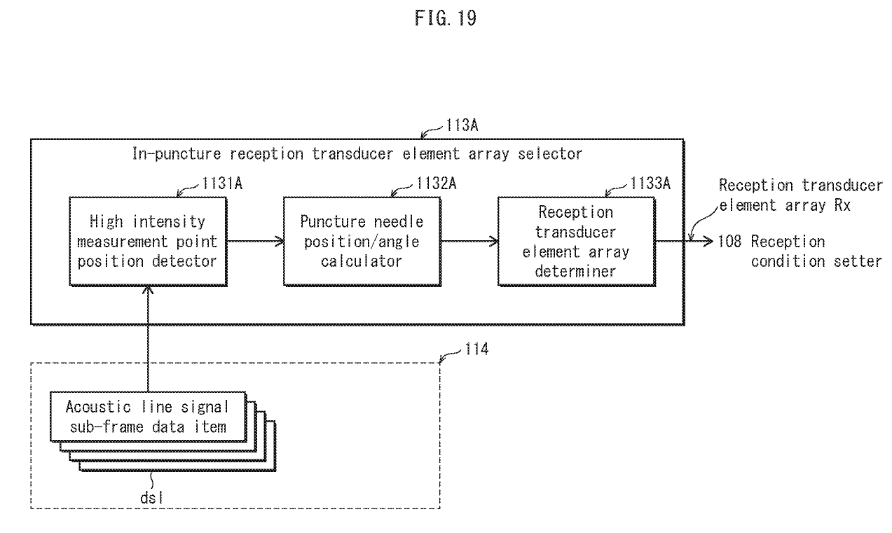

FIG. 19 illustrates functional blocks of the in-puncture reception transducer element array selector 113A. Each of FIGS. 20A through 20C is a schematic illustrating processing performed by the in-puncture reception transducer element array selector 113A, with an acoustic line signal sub-frame data item dsl illustrated over a corresponding position within an examination subject. As illustrated in FIG. 19, the in-puncture reception transducer element array selector 113A includes: a high intensity measurement point position detector 1131A; a puncture needle position/angle calculator 1132A; and a reception transducer element array determiner 1133A.

The high intensity measurement point position detector 1131A reads out one or more acoustic line signal sub-frame data items dsl from the data storage 114.

In the present embodiment, the high intensity measurement point position detector 1131A reads out, from the data storage 114, one or more acoustic line signal sub-frame data items dsl that have been generated in response to one or more transmission events preceding the processing-target transmission event. For example, the high intensity measurement point position detector 1131A may read out, from the data storage 114, one acoustic line signal sub-frame data item dsl that has been generated in response to the most recent transmission event having been performed by using the same transmission transducer element array Tx as the processing-target transmission event. Alternatively, the high intensity measurement point position detector 1131A may read out, from the data storage 114, a plurality of acoustic line signal sub-frame data items dsl that have been generated in response to transmission events performed before the processing-target transmission event and after the most recent transmission event having been performed by using the same transmission transducer element array Tx as the processing-target transmission event.

The high intensity measurement point position detector 1131A extracts, from each acoustic line signal sub-frame data item dsl that is read out, positions of measurement points (measurement points Pijh) producing acoustic line signals with intensities higher than or equal to the predetermined threshold. Specifically, from each acoustic line signal sub-frame data item dsl, the high intensity measurement point position detector 1131A extracts measurement points Pijh as illustrated in FIG. 20A. The measurement points Pijh are illustrated in the FIGS. 20A and 20B as black squares. As described above, the puncture needle 101b produces specular ultrasound reflection. Due to this, when an acoustic line signal sub-frame data item dsl having been generated in response to a transmission event includes an acoustic line signal based on ultrasound reflection from the puncture needle 101b, the acoustic line signal has extremely higher signal intensity than acoustic line signals based on ultrasound reflection from body tissues. The predetermined threshold is set to distinguish acoustic line signals based on ultrasound reflection from the puncture needle 101b from acoustic line signals based on ultrasound reflection from body tissues. As such, an image of the puncture needle 101b in an acoustic line signal frame data item dsl can be detected by detecting, in the acoustic line signal frame data item dsl, an array of measurement points Pijh producing acoustic line signals with intensities higher than or equal to the predetermined threshold.

The puncture needle position/angle calculator 1132A, by using an acoustic line signal sub-frame data item dsl including acoustic line signals with intensities higher than or equal to the predetermined threshold, analyzes the shape of the area where the measurement points Pijh producing such acoustic line signals exist in the acoustic line signal sub-frame data item dsl, as illustrated in FIG. 20B. When the area has a linear shape with a predetermined width, the puncture needle position/angle calculator 1132A determines that the area is a puncture needle imaging area 101bX imaging the puncture needle 101b, and geometrically calculates the position/angle of the puncture needle imaging area 101bX by performing processing such as thinning. Thus, the puncture needle position/angle calculator 1132A calculates the position/angle at which the puncture needle 101b has been imaged. The puncture needle position/angle calculator 1132A performs the specification of the puncture needle imaging area 101bX and the calculation of the position/angle at which the puncture needle 101b has been imaged for each acoustic line signal sub-frame data items dsl that is read out.

The reception transducer element array determiner 1133A, by using an acoustic line signal sub-frame data item dsl, geometrically calculates a position of a high intensity receive transducer element 101aX receiving ultrasound reflection from the puncture needle 101b located at the puncture needle imaging area 101bX. Here, for example, the reception transducer element array determiner 1133A may specify, as a high intensity receive transducer element 101aX, a transducer element 101a located at a position where ultrasound transmitted from the center position of the transducer elements 101a in the array-direction arrives after undergoing specular reflection at the puncture needle imaging area 101bX. Further, the reception transducer element array determiner 1133A selects, as a reception transducer element array Rx, a group of transducer elements 101a including the high intensity receive transducer element 101aX at the center thereof in the array direction.

When the puncture needle imaging area 101bX is specified in a plurality of acoustic line signal sub-frame data items dsl, the reception transducer element array determiner 1133A calculates the position of a high intensity receive transducer element 101aX in each of such acoustic line signal sub-frame data items dsl. In this case, it is preferable that the reception transducer element array determiner 1133A select, as a reception transducer element array Rx, a group of transducer elements 101a that includes all high intensity receive transducer elements 101aX having been calculated and that includes the center one of the high intensity receive transducer elements 101aX in the array direction at the center thereof in the array direction.

The in-puncture reception transducer element array selector 113A outputs information indicating the position of the reception transducer element array Rx it has selected to the reception condition setter 108. Meanwhile, when a puncture needle imaging area 101bX is not detected in any acoustic line signal sub-frame data item dsl, the in-puncture reception transducer element array selector 113A does not output any information indicating a position of a reception transducer element array Rx to the reception condition setter 108.

### <Operations>

The following describes the generation of acoustic line signal sub-frame data items dsl, performed by the ultrasound diagnostic device 100A. The generation of acoustic line signal sub-frame data items dsl by the ultrasound diagnostic device 100A differs from the same by the ultrasound diagnostic device 100 in terms of the processing for selecting a reception transducer element array Rx suitable for the generation of acoustic line signals from the puncture needle 101b. Thus, the following provides description of only this different processing.

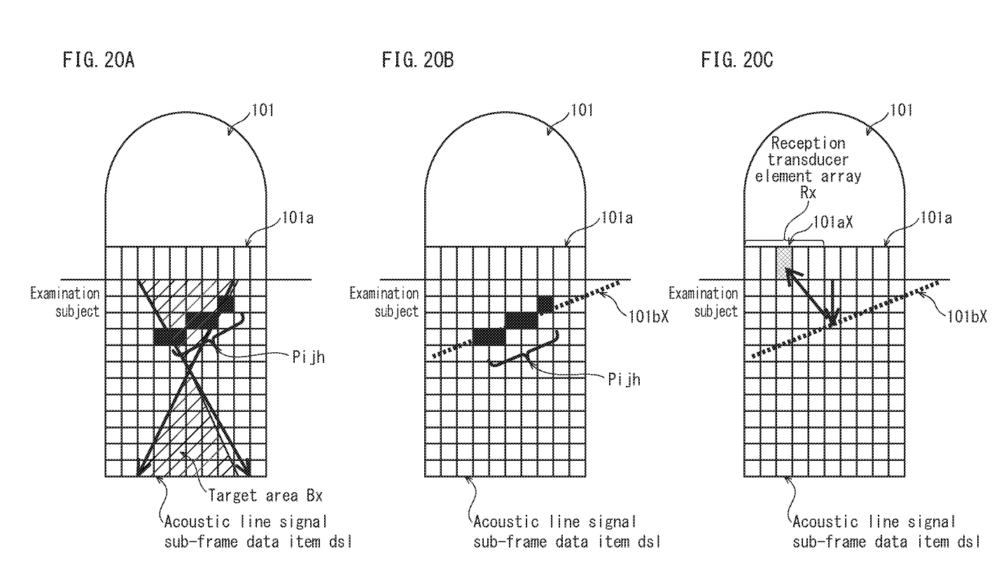

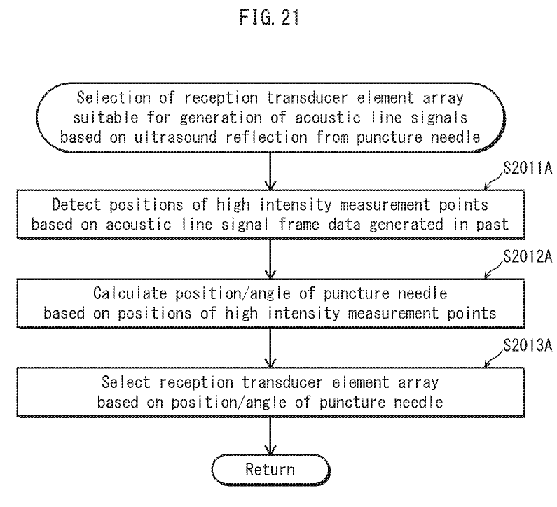

FIG. 21 is a flowchart illustrating the selection of a reception transducer element array Rx, performed by the in-puncture reception transducer element array selector 113A.

As described above, the in-puncture reception transducer element array selector 113A selects a reception transducer element array Rx based on puncture needle position/inclination angle calculated based on one or more acoustic line signal sub-frame data items dsl having been generated in response to one or more transmission events preceding the processing-target transmission event. For example, the in-puncture reception transducer element array selector 113A may perform the selection of a reception transducer element array Rx based on one acoustic line signal sub-frame data item dsl having been generated in response to the most recent transmission event performed by using the same transmission transducer element array Tx as the processing-target transmission event. Alternatively, the in-puncture reception transducer element array selector 113A may perform the selection of a reception transducer element array Rx based on a plurality of acoustic line signal sub-frame data items dsl having been generated in response to transmission events after the most recent transmission event performed by using the same transmission transducer element array Tx as the processing-target transmission event.

The in-puncture reception transducer element array selector 113A acquires one or more acoustic signal sub-frame data items rfl from the data storage 114.

In Step S2011A, the high intensity measurement point position detector 1131A extracts, from each acoustic line signal sub-frame data item dsl having been acquired, positions of measurement points Pijh producing acoustic line signals with intensities higher than or equal to the predetermined threshold.

In Step S2012A, the puncture needle position/angle calculator 1132A, in each acoustic line signal sub-frame data item dsl including a plurality of measurement points Pijh, analyzes the shape of the area where the measurement points Pijh exist in the acoustic line signal sub-frame data item dsl, specifies the area as a puncture needle imaging area 101bX when the area has a linear shape, and geometrically calculates the position/angle at which the puncture needle 101b has been imaged by calculating the position/angle of the puncture needle imaging area 101bX.

In Step S2013A, the reception transducer element array determiner 1133A selects a reception transducer element array Rx based on the detected position/angle at which the puncture needle 101b has been imaged. Specifically, the reception transducer element array determiner 1133A geometrically calculates a position of a high intensity receive transducer element 101aX receiving ultrasound reflection from the puncture needle 101b at the puncture needle imaging area 101bX acquired in Step S2012A, and selects a group of transducer element 101a including the high intensity receive transducer element 101aX at the center thereof in the array direction.

Alternatively, when selecting a reception transducer element array Rx based on puncture needle positions/inclination angles calculated based on a plurality of acoustic line signal sub-frame data items dsl having been generated in response to a plurality of transmission events preceding the current transmission event, processing may be performed as follows. When a puncture needle imaging area 101bX has been specified in each of a plurality of acoustic line signal sub-frame data items dsl, the reception transducer element array determiner 1133A may select, as a reception transducer element array Rx, a group of transducer elements that includes that includes all of the high intensity receive transducer elements 101aX having been specified based on the acoustic line signal sub-frame data items dsl and that includes the center one of the high intensity receive transducer elements 101aX in the array direction at the center thereof in the array direction.

The in-puncture reception transducer element array selector 113A outputs information indicating the position of the reception transducer element array Rx so selected to the reception condition setter 108. Following this, a combined acoustic line signal frame data item ds is generated through the relevant processes illustrated in FIG. 15.

### <Effects>

As described up to this point, the ultrasound diagnostic device 100A is capable of specifying, as a reception transducer element array Rx, a group of transducer elements 101a whose receives signals rf based on ultrasound reflection from the puncture needle 101b have high intensity. Specifically, the in-puncture reception transducer element array selector 113A of the ultrasound diagnostic device 100A extracts a linear area composed of measurement points producing acoustic line signals with high intensity in an acoustic line signal sub-frame data item dsl, specifies the position/inclination angle at which the puncture needle 101b has been imaged based on the linear area, and selects a reception transducer element array Rx based on the specified position/inclination angle.

With this structure, the ultrasound diagnostic device 100A, similar to the ultrasound diagnostic device 100 pertaining to embodiment 1, is capable of producing ultrasound images imaging a puncture needle with high visible perceptibility, with reception beam forming utilizing the synthetic aperture method. Specifically, the ultrasound diagnostic device 100A is capable of calculating puncture needle angle/inclination angle with high precision, by performing the calculation of angle/inclination angle at which the puncture needle has been imaged by using an acoustic line signal sub-frame data item dsl, which is generated through delay-and-sum processing and thus has a high signal S/N ratio.

Further, an acoustic line signal sub-frame data item dsl having been generated in response to a transmission event may include a linear area composed of measurement points producing acoustic line signals with high signal intensity. This linear area is indicative of the fact that ultrasound reflection from the puncture needle 101b has been received by the reception transducer element array Rx for the transmission event. As such, specifying a high intensity receive transducer element 101aX from the detected linear area and selecting a group of transducer elements 101a including the high intensity receive transducer element 101aX at the center thereof in the array direction as a reception transducer element array Rx for another transmission event increases the possibility of ultrasound reflection from the puncture needle 101b being received in the another transmission event.

Note that the method described in embodiment 2 is a non-limiting example of a method of selecting a reception transducer element array Rx based on acoustic line signals. That is, the selection of a reception transducer element array Rx can be performed by using other methods as long as puncture needle position is detected based on acoustic line signal and a reception transducer element array Rx is selected based on the detected position.

### Embodiment 3

The following describes an ultrasound diagnostic device 100B pertaining to embodiment 3. The ultrasound diagnostic device 100A pertaining to embodiment 2 includes the in-puncture reception transducer element array selector 113A, which specifies the position/inclination angle at which the puncture needle has been imaged based on the characteristics of acoustic line signals in one or more acoustic line signal sub-frame data item dsl, and selects a reception transducer element array Rx based on the specified position/inclination angle. However, various other methods can be used as necessary or desired for specifying puncture needle position/inclination angle. Embodiment 3 describes one example of such methods.

The ultrasound diagnostic device 100B differs from the ultrasound diagnostic device 100A pertaining to embodiment 2 for including a probe interface acquiring, from the probe 101, probe identification information identifying the probe 101, and an in-puncture reception transducer element array selector that specifies the position/inclination angle of the puncture needle 101b based on the probe identification information and selects a reception transducer element array Rx based on the specified position/inclination angle.

### <Structure>

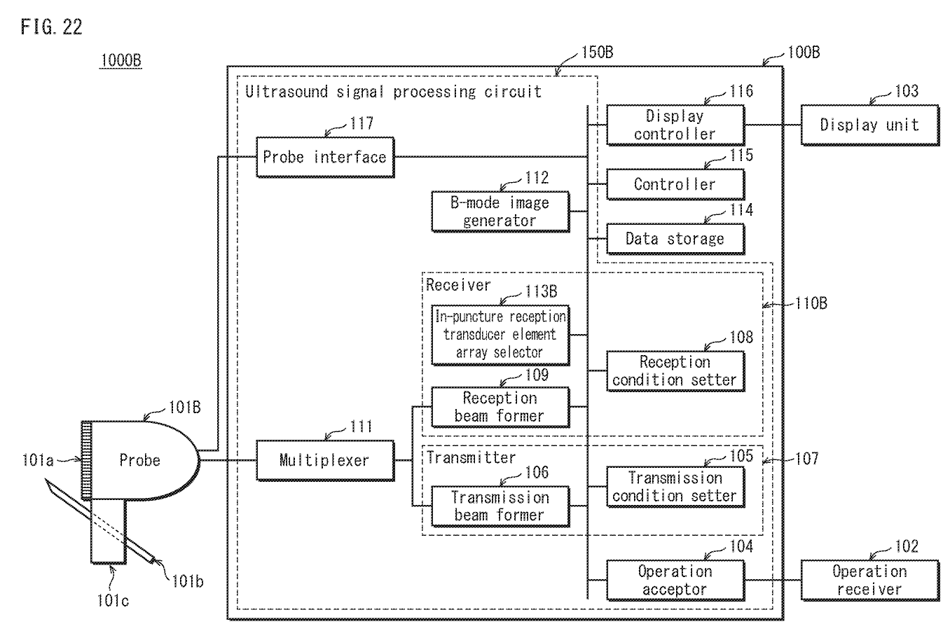

The following describes the structure of the ultrasound diagnostic device 100B. FIG. 22 illustrates functional blocks of an ultrasound diagnostic system 1000B including the ultrasound diagnostic device 100B. The ultrasound diagnostic device 100B differs from the ultrasound diagnostic device 100 for including an in-puncture reception transducer element array selector 113B and a probe interface 117, as illustrated in FIG. 22. The following description focuses on the in-puncture reception transducer element array selector 113B and the probe interface 117. Since the rest of the components of the ultrasound diagnostic device 100B other than the in-puncture reception transducer element array selector 113B and the probe interface 117 are similar to those in the ultrasound diagnostic device 100, description is not provided of the rest of the components of the ultrasound diagnostic device 100B.

Upon connection of the probe 101 to the ultrasound diagnostic device 100B, the probe interface 117 acquires, from the probe 101, probe identification information identifying the probe 101. The probe identification information is information unique to the probe 101, and enables specifying the model and specifications of the probe 101 as well as enabling individually identifying the specific probe 101. The probe interface 117 outputs the probe identification information so acquired to the in-puncture reception transducer element array selector 113B.

The in-puncture reception transducer element array selector 113B is a circuit that specifies the position/inclination angle of the puncture needle 101b based on the probe identification information that it acquires from the probe interface 117, and selects a reception transducer element array Rx based on the specified position/inclination angle.

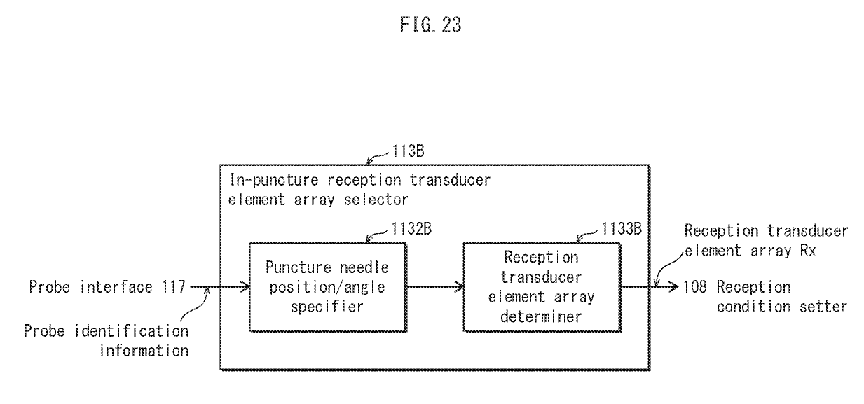

FIG. 23 illustrates functional blocks of the in-puncture reception transducer element array selector 113B. Each of FIGS. 24A and 24B is a schematic illustrating processing by the in-puncture reception transducer element array selector 113B. As illustrated in FIG. 23, the in-puncture reception transducer element array selector 113B includes a puncture needle position/angle specifier 1132B and a reception transducer element array determiner 1133B.

As illustrated in FIG. 24A, the probe 101 has the puncture guide 101c attached thereto, with the puncture guide 101c holding the puncture needle 101b. As already described above, the puncture guide 101c has an undepicted guide member that, when the probe 101 is put in contact with the skin surface of the examination subject, guides the puncture needle 101b into the examination subject at a predetermined insertion angle. Usually, every probe has a specific puncture guide compatible therewith, and the puncture guide defines where the puncture needle is attached to the probe and the insertion angle of the puncture needle with respect to examination subject. Thus, acquisition of the probe identification information of the probe 101 allows specifying the position np of the puncture needle 101b relative to the transducer elements 101a of the probe 101, and the inclination angle na of the puncture needle 101b relative to the surface of the array of transducer elements 101a of the probe 101.

The puncture needle position/angle specifier 1132B is capable of specifying the position np and the inclination angle na of the puncture needle 101b based on the probe identification information that it acquires.

Meanwhile, as illustrated in FIG. 24A, the reception transducer element array determiner 1133B geometrically calculates a position of a high intensity receive transducer element 101aX receiving ultrasound reflection from the puncture needle 101b, based on information indicating the position np and the inclination angle na of the puncture needle 101b that it acquires. Here, by using a method similar to that described in embodiment 2, the reception transducer element array determiner 1133B may specify a puncture needle imaging area 101bX in an acoustic line signal sub-frame data item dsl, and geometrically calculate the position of a high intensity receive transducer element 101aX based on the position of the puncture needle imaging area 101bX, as illustrated in FIG. 24B. Further, the reception transducer element array determiner 1133C selects, as a reception transducer element array Rx, a group of transducer elements 101a including the high intensity receive transducer element 101aX at the center thereof in the array direction. The reception transducer element array determiner 1133C outputs information indicating the position of the reception transducer element array Rx it has selected to the reception condition setter 108.

### <Operations>

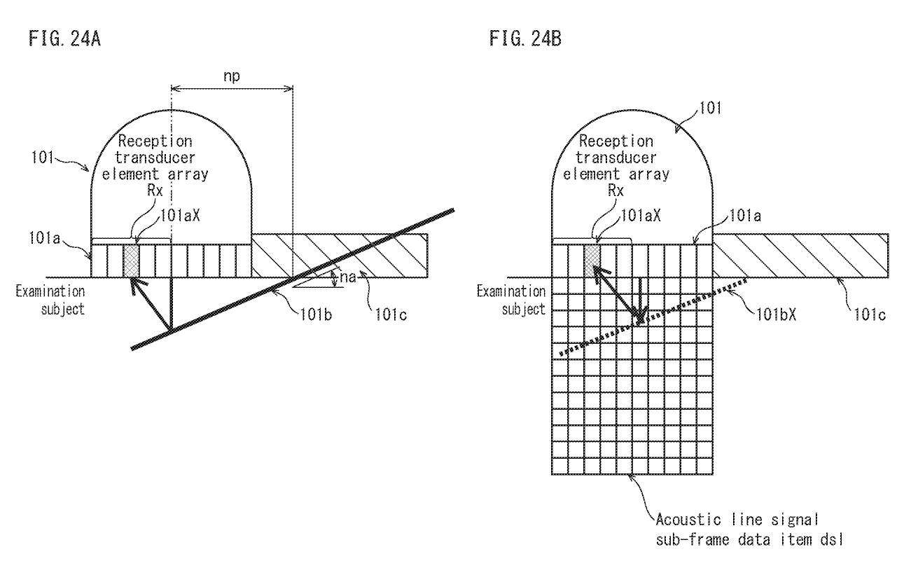

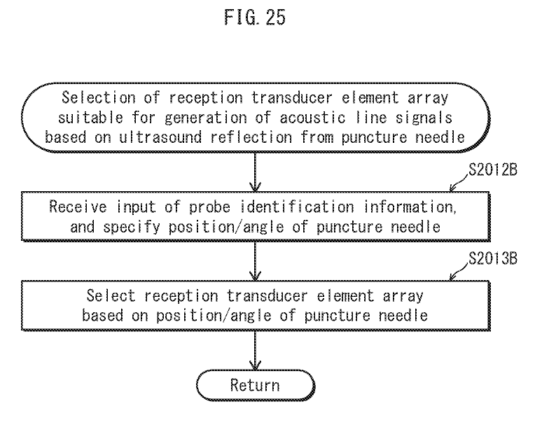

The following describes the generation of acoustic line signal sub-frame data items dsl, performed by the ultrasound diagnostic device 100B. The following describes the processing (illustrated in FIG. 25) by the ultrasound diagnostic device 100C of selecting a reception transducer element array Rx suitable for the generation of acoustic line signals from the puncture needle 101b, which differs from that of the ultrasound diagnostic device 100.

The in-puncture reception transducer element array selector 113B reads out one or more receive signal sub-frame data items rfl from the data storage 114.

The puncture needle position/angle specifier 1132B acquires, from the probe interface 117, probe identification information identifying the probe 101 connected to the ultrasound diagnostic device 100B, and specifies the position np and the inclination angle na of the puncture needle 101b based on the probe identification information (Step S2012B). The reception transducer element array determiner 1133B geometrically calculates the position of a high intensity receive transducer element 101aX based on the acquired position np and the acquired angle na of the puncture needle 101b, and selects, as a reception transducer element array Rx, a group of transducer elements 101a including the high intensity receive transducer element 101aX at the center thereof in the array direction (Step S2013B). The in-puncture reception transducer element array selector 113B outputs information indicating the position of the reception transducer element array Rx so selected to the reception condition setter 108. Following this, a combined acoustic line signal frame data item ds is generated through the relevant processes illustrated in FIG. 15.

### <Effects>

As described up to this point, the ultrasound diagnostic device 100B pertaining to embodiment 3 includes the probe interface 117 and the in-puncture reception transducer element array selector 113B. The probe interface 117 acquires probe identification information from the probe 101. The in-puncture reception transducer element array selector 113B specifies the position np and the inclination angle na of the puncture needle 101b based on the probe identification information, and selects a reception transducer element array Rx based on the position np and the inclination angle na.

The ultrasound diagnostic device 100B is capable of producing ultrasound images imaging a puncture needle with high visual perceptibility. Specifically, the ultrasound diagnostic device 100B is capable of specifying puncture needle position/inclination angle by using a probe interface, and thus, is capable of specifying puncture needle position/inclination angle with a simple structure, without having to execute detection of puncture needle position/inclination angle based on acoustic line signals, which typically has great processing load.

### Embodiment 4

The following describes an ultrasound diagnostic device 100C pertaining to embodiment 4. As described above, the ultrasound diagnostic device 100B pertaining to embodiment 3 includes the probe interface 117 and the in-puncture reception transducer element array selector 113B. The probe interface 117 acquires probe identification information from the probe 101, and the in-puncture reception transducer element array selector 113B specifies the position/inclination angle of the puncture needle 101b based on the probe identification information. However, various other methods can be used as necessary or desired for specifying puncture needle position/inclination angle. Embodiment 4 describes one example of such methods.

The ultrasound diagnostic device 100C is configured to be connectable to the operation receiver 102, which receives input operations performed by an examiner. This is similar to embodiments 1, 2, and 3. However, embodiment 4 differs from embodiment 3 in that the ultrasound diagnostic device 100C includes an in-puncture reception transducer element array selector 113C, and the in-puncture reception transducer element array selector 113C, when information indicating the position np and the inclination angle na of a puncture needle is input to the operation receiver 102, selects a reception transducer element array Rx based on the information that is input.

### <Structure>

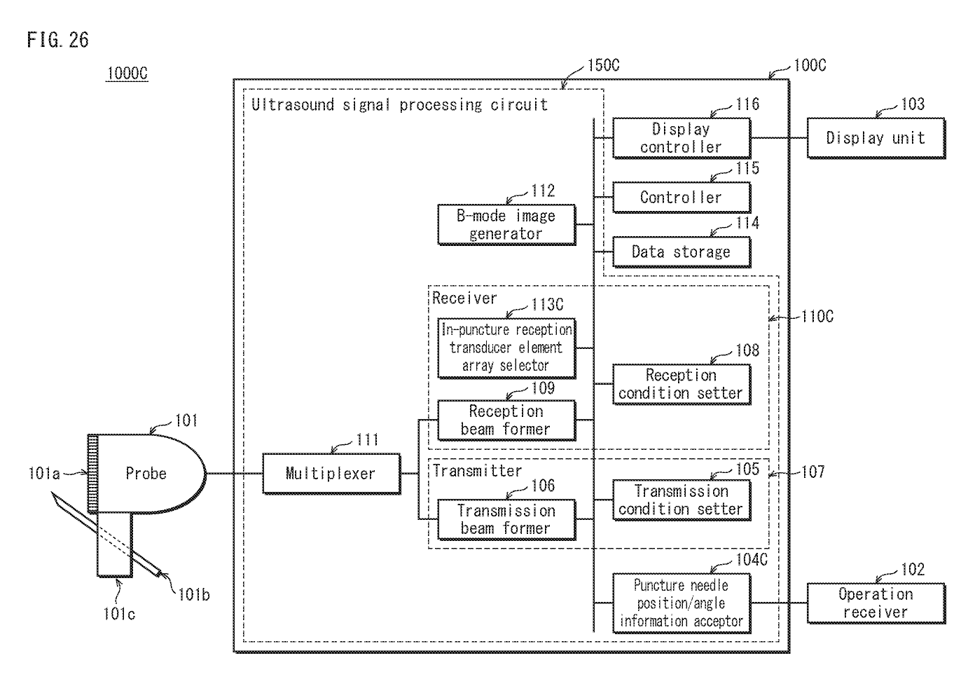

The following describes the structure of the ultrasound diagnostic device 100C. FIG. 26 illustrates functional blocks of an ultrasound diagnostic system 1000C including the ultrasound diagnostic device 100C. The ultrasound diagnostic device 100C differs from the ultrasound diagnostic device 100 for including an in-puncture reception transducer element array selector 113C and a puncture needle position/angle information acceptor 104C, as illustrated in FIG. 26. The following description focuses on the in-puncture reception transducer element array selector 113C and the puncture needle position/angle information acceptor 104C. Since the rest of the components of the ultrasound diagnostic device 100C other than the in-puncture reception transducer element array selector 113C and the puncture needle position/angle information acceptor 104C are similar to those in the ultrasound diagnostic device 100, description is not provided of the rest of the components of the ultrasound diagnostic device 100C.

The puncture needle position/angle information acceptor 104C is similar to the operation acceptor 104 of the ultrasound diagnostic device 100 for having the function of receiving, from the operation receiver 102, information indicating a manual input operation that the examiner has performed on the operation receiver 102. The input operation performed by the examiner may be, but is not limited to, an input operation for changing settings of the ultrasound diagnostic device 100C or an input operation for controlling the ultrasound diagnostic device 100C. In addition, the puncture needle position/angle information acceptor 104C has the function of receiving, from the operation receiver 102, information indicating the insertion angle of the puncture needle 101b with respect to the body tissues of the examination subject, the position of the puncture needle 101b relative to the transducer elements 101a, etc., when the examiner has performed a manual input operation on the operation receiver 102 to input such information. Here, the operation receiver 102 may be configured to enable the examiner to input such information when the examiner provides the operation receiver 102 with input activating an operation mode for puncture needle insertion. The puncture needle position/angle information acceptor 104C outputs the information that it receives from the operation receiver 102 to the in-puncture reception transducer element array selector 113C, via the controller 115.

The in-puncture reception transducer element array selector 113C is a circuit that selects a reception transducer element array Rx based on puncture needle position/angle information that is acquires.

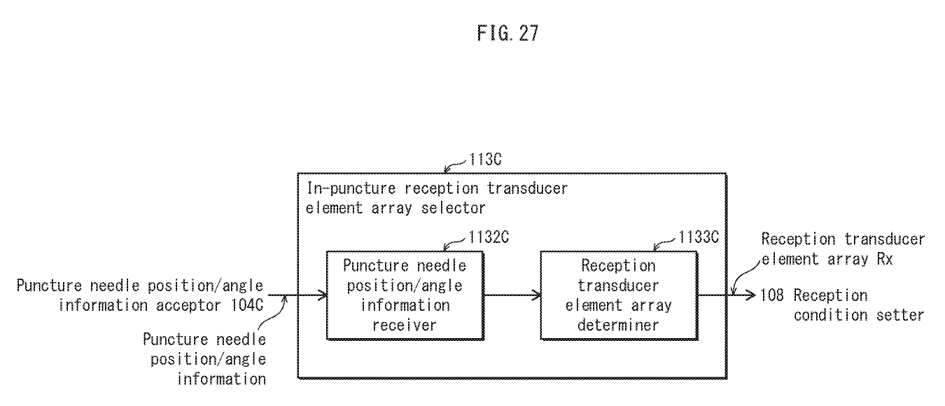

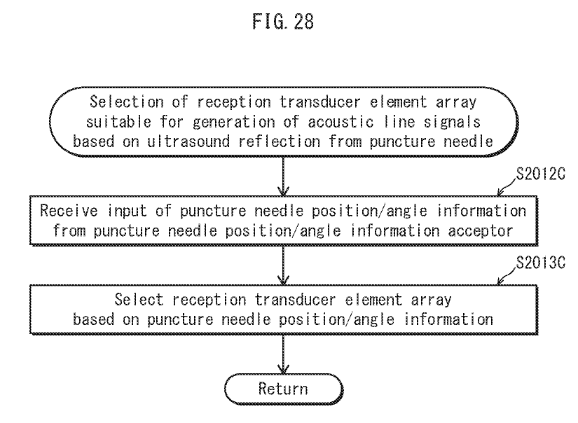

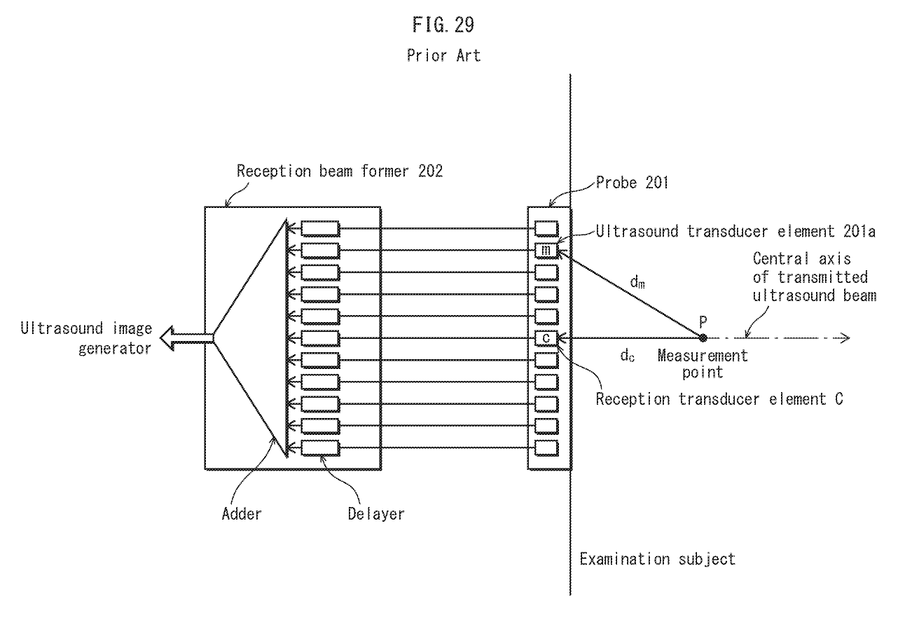

FIG. 27 illustrates functional blocks of the in-puncture reception transducer element array selector 113C. Each of FIGS. 28A and 28B is a schematic illustrating processing by the in-puncture reception transducer element array selector 113C. As illustrated in FIG. 27, the in-puncture reception transducer element array selector 113C includes a puncture needle position/angle information receiver 1132C and a reception transducer element array determiner 1133C.

The puncture needle position/angle information receiver 1132C receives the position np and the inclination angle na of the puncture needle 101b from the puncture needle position/angle information acceptor 104C.

As illustrated in FIG. 24A, the reception transducer element array determiner 1133C geometrically calculates a position of a high intensity receive transducer element 101aX receiving ultrasound reflection from the puncture needle 101b, based on information indicating the position np and the inclination angle na of the puncture needle 101b that it acquires. That is, the reception transducer element array determiner 1133C geometrically calculates the position of a high intensity receive transducer element 101aX according to a method similar to that described in embodiment 3. Further, the reception transducer element array determiner 1133C selects, as a reception transducer element array Rx, a group of transducer elements 101a including the high intensity receive transducer element 101aX at the center thereof in the array direction. The reception transducer element array determiner 1133C outputs information indicating the position of the reception transducer element array Rx it has selected to the reception condition setter 108.

### <Operations>

The following describes the generation of acoustic line signal sub-frame data items dsl, performed by the ultrasound diagnostic device 100C. The following describes the processing (illustrated in FIG. 28) by the ultrasound diagnostic device 100C of selecting a reception transducer element array Rx suitable for the generation of acoustic line signals from the puncture needle 101b, which differs from that of the ultrasound diagnostic device 100.

The in-puncture reception transducer element array selector 113C reads out one or more receive signal sub-frame data items rfl from the data storage 114. The puncture needle position/angle information receiver 1132C receives the position np and the inclination angle na of the puncture needle 101b from the puncture needle position/angle information acceptor 104C (Step S2012C). The reception transducer element array determiner 1133C geometrically calculates the position of a high intensity receive transducer element 101aX based on the position np and the angle na of the puncture needle 101b that it acquires, and selects, as a reception transducer element array Rx, a group of transducer elements 101a including the high intensity receive transducer element 101aX at the center thereof in the array direction (Step S2013C). The in-puncture reception transducer element array selector 113C outputs information indicating the position of the reception transducer element array Rx so selected to the reception condition setter 108. Following this, a combined acoustic line signal frame data item ds is generated through the relevant processes illustrated in FIG. 15.

### <Effects>

As described up to this point, the ultrasound diagnostic device 100C is configured to be connectable to the operation receiver 102, which receives input operations performed by an examiner. Further, the ultrasound diagnostic device 100C includes an in-puncture reception transducer element array selector 113C. The in-puncture reception transducer element array selector 113C, when information indicating the position np and the inclination angle na of a puncture needle is input to the operation receiver 102, selects a reception transducer element array Rx based on the information that is input.

The ultrasound diagnostic device 100C is capable of producing ultrasound images imaging a puncture needle appear with high visual perceptibility. Specifically, the ultrasound diagnostic device 100C is capable of specifying puncture needle position/inclination angle without a probe interface, and thus with an even simpler structure. Specifically, the ultrasound diagnostic device 100C is capable of specifying puncture needle position/inclination angle by including the puncture needle position/angle information acceptor 104C, which receives information indicating puncture needle position/inclination angle that has been input on the operation receiver 102.

### <Other Modifications>

Up to this point, the technology pertaining to the present disclosure has been described based on specific embodiments thereof. However, the embodiments are non-limiting examples of application of the technology pertaining to the present disclosure, and thus, the technology pertaining to the present disclosure shall be construed to encompass the following exemplar modifications.

For example, the technology pertaining to the present disclosure may be implemented by using a computer system including a memory storing a computer program and a microprocessor operating based on the computer program. For example, the computer system may store a computer program of a diagnosis method of an ultrasound diagnostic device pertaining to the technology of the present disclosure, and the computer system may operate in accordance with the computer program or may provide instructions in accordance with the computer program to various components connected thereto.

Further, the technology pertaining to the present disclosure may be implemented by implementing a part of or the entirety of an ultrasound diagnostic device described above, or a part of or an entirety of an beam former described above by using a computer system including a microprocessor, a recording medium such as a ROM or a RAM, and a hard disk unit. In this implementation, a computer program achieving the same operations as a device described above is stored to the RAM or the hard disk unit. Further, in this implementation, a device achieves its functions by the microprocessor operating in accordance with the computer program.

Further, the technology pertaining to the present disclosure may be implemented by implementing some or all components included in a device described above by using one system LSI (large scale integration). A system LSI is an ultra-multifunctional LSI manufactured by integrating multiple components onto one chip. Specifically, a system LSI is a computer system including a microprocessor, a ROM, a RAM, and the like. Further, each component may be separately implemented by using one chip, or some or all components may be implemented by using one chip. Note that LSIs are referred to by using different names, depending upon the level of integration achieved thereby. Such names include IC, system LSI, super LSI, and ultra LSI. In this implementation, a computer program achieving the same operations as a device described above is stored to the RAM. Further, in this implementation, the system LSI achieves its functions by the microprocessor operating in accordance with the computer program. For example, the technology pertaining to the present disclosure encompasses a form of implementation where an LSI stores a beam forming method pertaining to the present disclosure as a program, the LSI is inserted into a computer, and the computer executes the program (i.e., the beam forming method pertaining to the present disclosure).

Note that integration of circuits may be achieved by a dedicated circuit or a general purpose processor, in addition to being achievable by using an LSI as discussed above. Further, a Field Programmable Gate Array (FPGA), which is programmable after manufacturing, or a reconfigurable processor, which allows reconfiguration of the connection and setting of circuit cells inside the LSI, may be used.

Furthermore, if technology for circuit integration that replaces LSIs emerges, owing to advances in semiconductor technology or to another derivative technology, the integration of functional blocks may naturally be accomplished using such technology.

Further, some or all functions of an ultrasound diagnostic device discussed in the embodiments may be implemented by a processor such as a CPU executing a program. Further, the technology pertaining to the present disclosure may be implemented by using a non-transitory computer-readable recording medium having recorded thereon a program causing execution of a diagnostic method and a beam forming method of an ultrasound diagnostic device described above. Further, execution of the program by another independent computer system may be achieved by transferring the program by recording the program or a signal onto a recording medium. Naturally, the program may be distributed via means of transmission media such as the internet.

Each of the ultrasound diagnostic devices pertaining to the embodiments includes the data storage, which is a recording device. However, the recording device need not be included in the ultrasound diagnostic devices, and may be implemented by using a semiconductor memory, a hard disk drive, an optical disk drive, a magnetic storage device, or the like connected to the ultrasound diagnostic devices from the outside.

Further, the functional blocks illustrated in the block diagrams are mere examples of possible functional blocks. That is, a plurality of functional blocks illustrated in the block diagrams may be combined to form one functional block, a given functional block illustrated in the block diagrams may be divided into a plurality of functional blocks, and a function of a given functional block illustrated in the block diagrams may be transferred to another functional block. Further, with regards to multiple functional blocks having similar functions, such functional blocks may be implemented by one piece of hardware or software executing such functions in parallel or by applying time division.

Further, the above-described order in which steps of processing are executed is a non-limiting example, among multiple possible orders, that is used for the sole sake of providing specific description of the technology pertaining to the present disclosure. Further, some of the steps of processing described above may be executed simultaneously (in parallel).

Further, in the embodiments, description is provided that the ultrasound diagnostic devices may have a probe and a display attached thereto. However, the ultrasound diagnostic devices may include a probe and a display therein.

Further, in the embodiments, the probe includes a plurality of piezoelectric transducer elements arrayed along one direction. However, the probe may have a different structure. For example, the probe may include a plurality of piezoelectric transducer elements arrayed two-dimensionally. Alternatively, the probe may be a swingable probe including a plurality of swingable transducer elements (i.e., transducer elements that can be caused to swing by mechanical means) arrayed along one direction, which enables acquisition of three-dimensional tomographic images. Further, probes of different types may be selected and used depending upon the examination to be performed. For example, when using a probe including piezoelectric transducer elements arrayed two-dimensionally, supplying different piezoelectric transducer elements with voltages at different timings or with voltages with different values achieves controlling the irradiation position, direction, etc., of the ultrasound beam to be transmitted.

Further, the probe may be provided with some of the functions of the transmitter/receiver. For example, the probe may be capable of generating a transmission electric signal based on a control signal that the transmitter/receiver outputs to cause generation of a transmission electric signal, and of converting the transmission electronic signal into ultrasound. In addition, the probe may be capable of converting ultrasound reflection into receive signals, and of generating acoustic line signals based on the receive signals.

The transmitter 107 and the receiver 110 in the ultrasound diagnostic devices pertaining to the embodiments need not have the respective structures described in the embodiments, and may be provided with different structures as required. For example, in the embodiments, the transmitter 107 sets, for each transmission event, a group of some of the transducer elements 101a of the probe 101 as a transmission transducer element array, and performs transmission events repeatedly while gradually shifting the transmission transducer element array in the array direction each time, to cause all of the transducer elements 101a of the probe 101 to perform ultrasound transmission. However, the transmitter 107 may cause all the transducer elements 101a of the probe 101 to perform ultrasound transmission all at once. This modification achieves receiving ultrasound reflection from the entire ultrasound main irradiation area through one transmission event, and thus, makes unnecessary performing transmission events repeatedly.

Further, in the embodiments, the receiver 110 causes, for each transmission event, all of the transducer elements 101a of the probe 101 to each receive ultrasound reflection. However, the receiver 110 may set, for each transmission event, a group of some of the transducer elements 101a of the probe 101 as a receive transducer element array, and perform reception of ultrasound reflection repeatedly, while gradually shifting the receive transducer element array in the array direction each time, such that all of the transducer elements 101a of the probe 101 each receive ultrasound reflection. This achieves, for each transmission event, generating sequences of receive signals by using only ultrasound reflection received from the vicinity of the transmitted ultrasound, and generating acoustic line signals based on such sequences of receive signals in the target area. Thus, this reduces the processing load of the generation of acoustic line signals for each transmission event, and further improves signal resolution in the time domain.

Further, the area within which measurement points are distributed (i.e., the target area) need not have the shape described in the embodiments, as long as target areas are set inside ultrasound main irradiation areas. For example, a target area may be an hourglass-shaped area whose base has a length corresponding to the length of the reception transducer element array and whose central axis passes through the center position of the reception transducer element array in the array direction. Alternatively, a target area may be a rectangular, belt-shaped area having a width corresponding to the widths of at least two transducer elements and whose central axis passes through the center position of the reception transducer element array in the array direction and is perpendicular to the reception transducer element array. When making such modifications, the central axis of the target area need not pass through the center position of the reception transducer element array in the array direction.

Further, in embodiment 2, the receiver 110 selects a reception transducer element array Rx based on the position np and the inclination angle na of the puncture needle 101b, which are calculated based on an acoustic line signal sub-frame data item dsl having been generated in response to a transmission event preceding the processing-target transmission event. Alternatively, the receiver 110 may first generate a provisional acoustic line signal sub-frame data item dsl for the processing-target transmission event, then select a reception transducer element array Rx based on the position and the inclination angle of the puncture needle 101b calculated based on the provisional acoustic line signal sub-frame data item dsl, and generate an acoustic line signal sub-frame data item dsl for the processing-target transmission event by performing delay-and-sum processing by using the sequences of receive signals corresponding to the transducer elements 101a in the selected reception transducer element array Rx. Here, the receiver 110 may generate the provisional acoustic line signal sub-frame data item dsl by selecting a provisional reception transducer element array Rx based on a certain rule (e.g., the transmission transducer element array for the processing-target transmission event may be selected as the provisional reception transducer element array Rx) and performing delay-and-sum processing by using sequences of receive signals corresponding to the transducer elements 101a in the provisional reception transducer element array Rx. This modification enables detection of the position np and the inclination angle na of the puncture needle 101b even when no transmission event has been performed preceding the processing-target transmission event (e.g., the processing-target transmission event is the first transmission event being performed), as well as producing ultrasound images imaging a puncture needle appear with high visual perceptibility.

Further, at least some of the functions of the ultrasound diagnostic devices pertaining to the embodiments and the modifications may be combined with functions of other ones of the ultrasound diagnostic devices pertaining to the embodiments and the modifications. Further, the values used above are non-limiting examples used for the sole sake of providing specific description of the technology pertaining to the present disclosure, and may be replaced with other values. Further, the technology pertaining to the present disclosure should be construed as encompassing various modifications that a skilled artisan would arrive at based on the embodiments describe above.

### <<Summary>>

One aspect of the present disclosure is an ultrasound diagnostic device to which an ultrasound probe having a plurality of transducer elements arrayed along an array direction is connectable, wherein the ultrasound diagnostic device: (i) repeatedly performs transmission events, each by selecting a first group of transducer elements from among the plurality of transducer elements and causing the first group to transmit ultrasound towards an examination subject having a puncture needle inserted therein; (ii) for each of the transmission events, generates a sub-frame data item based on ultrasound reflection received in response to the transmission event, the sub-frame data item including acoustic line signals; and (iii) combines sub-frame data items for the transmission events to generate a frame data item, the frame data item including combined acoustic line signals each being an aggregate of ones of the acoustic line signals, and the ultrasound diagnostic device includes an ultrasound signal processing circuit operating as: a transmitter that performs each of the transmission events by selecting the first group and causing the first group to transmit ultrasound focusing inside the examination subject, the transmitter gradually shifting the first group in the array direction between transmission events; and a receiver that: (i) for each of the transmission events, selects at least some of the plurality of transducer elements and generates a sequence of receive signals for each of the selected transducer elements based on ultrasound reflection that the selected transducer element has received from the examination subject in response to the transmission event; (ii) selects a second group of transducer elements, the transducer elements in the second group being ones among the plurality of transducer elements whose receive signals based on ultrasound reflection from the puncture needle have high intensity; (iii) for each of the transmission events, sets a target area for generating the sub-frame data item inside a virtual area of the examination subject that receives ultrasound transmitted in the transmission event, and generates the sub-frame data item by performing, for each of a plurality of measurement points in the target area, delay-and-sum processing on receive signals, for the transducer elements in the second group, based on ultrasound reflection from the measurement point; and (iv) generates the frame data item by combining the sub-frame data items for the transmission events.

With this structure, the ultrasound diagnostic device is capable of producing ultrasound images imaging a puncture needle with high visible perceptibility, with reception beam forming utilizing the synthetic aperture method. Specifically, the ultrasound diagnostic device utilizes the synthetic aperture method and repeatedly performs transmission events while gradually shifting the transmission transducer element array in the array direction. Further, the ultrasound diagnostic device performs delay-and-sum processing for each transmission event by using a reception transducer element array composed of reception transducer elements whose receive signals based on ultrasound reflection from the puncture needle have high intensity.

When performing ultrasound examination by using the ultrasound diagnostic device with the puncture needle inserted in the examination subject, signals based on ultrasound reflection from the puncture needle have extremely higher intensity than signals based on ultrasound reflection from body tissues since the puncture needle produces specular ultrasound reflection whereas body tissues mainly produce diffuse ultrasound reflection. The ultrasound diagnostic device distinguishes signals based on ultrasound reflection from the puncture needle from signals based on ultrasound reflection from body tissues by using a predetermined threshold. Specifically, the ultrasound diagnostic device detects a transducer element whose receive signal has intensity higher than or equal to the predetermined threshold, specifies such a transducer element as a transducer element having received ultrasound reflection from the puncture needle, and selects a group of transducer elements including such a transducer element as a reception transducer element array. Thus, the ultrasound diagnostic device is capable of selecting, as a reception transducer element array, a group of transducer elements whose receive signals based on ultrasound reflection from the puncture needle have relatively high intensity. The ultrasound diagnostic device is capable of producing ultrasound images imaging the puncture needle with high visual perceptibility by performing, for each measurement point in a target area, delay-and-sum processing on receive signals corresponding to the reception transducer elements of a reception transducer element array selected in such a manner to generate an acoustic line signal sub-frame data item for the target area.

Meanwhile, the generation of acoustic line signals for the target area, performed by using the selected reception transducer element array, also allows generating acoustic line signals based on ultrasound reflection from body tissues. This is since, even when a group of transducer elements whose receive signals based on ultrasound reflection from the puncture needle have high intensity is selected as a reception transducer element array, the transducer elements included in the selected reception transducer element array also have received ultrasound reflection from body tissues of the examination subject due to body tissues mainly producing diffuse ultrasound reflection.

Another aspect of the present disclosure is an ultrasound diagnostic device with any structure described above, wherein the receiver may detect, from among the generated sequences of receive signals, a sequence of receive signals including a high intensity receive signal, specify a high intensity transducer element, among the selected transducer elements, corresponding to the high intensity receive signal, and select a group of transducer elements including the high intensity transducer element as the second group.

With this structure, the ultrasound diagnostic device is capable of selecting, as a reception transducer element array, a group of transducer elements whose receive signals based on ultrasound reflection from the puncture needle have high intensities with a simple circuit structure, by performing simple processing that does not require prior execution of delay-and-sum processing, which typically has great processing load.

Another aspect of the present disclosure is an ultrasound diagnostic device with any structure described above, wherein the receiver may specify two or more high intensity transducer elements based on different ones of the transmission events, and select a group of transducer elements including the two or more high intensity transducer elements as the second group.

With this structure, the ultrasound diagnostic device is capable of performing reception beam forming utilizing the synthetic aperture method, where the transmission transducer element array is gradually shifted in the array direction between different transmission events, by using a reception transducer element array that enables efficiently generating acoustic line signals based on ultrasound reflection from the puncture needle across multiple transmission events, selected independent from the positions of the transmission transducer element arrays for the transmission events.

Another aspect of the present disclosure is an ultrasound diagnostic device with any structure described above, wherein from at least one of the sub-frame data items, the receiver may extract a linear area composed of acoustic line signals with high intensity, calculate a position and an inclination angle at which the puncture needle is imaged based on the linear area, and select the second group based on the position and the inclination angle.

With this structure, the ultrasound diagnostic device is capable of calculating puncture needle angle/inclination angle with high precision, for performing the calculation of angle/inclination angle at which the puncture needle has been imaged in an ultrasound image by using an acoustic line signal sub-frame data item dsl, which is generated through delay-and-sum processing and thus has a high signal S/N ratio.

Another aspect of the present disclosure is an ultrasound diagnostic device with any structure described above, wherein the at least one of the sub-frame data items may be a sub-frame data item having been generated for a transmission event preceding a processing-target transmission event.

With this structure, the ultrasound diagnostic device, when an acoustic line signal sub-frame data item having been generated in response to a transmission event includes a linear area composed of acoustic line signals with high signal intensity, selects a group of transducer elements including a high intensity receive transducer element specified based on the detected linear area at the center thereof as a reception transducer element array and thus increases the possibility of ultrasound reflection from the puncture needle being received in a subsequent transmission event. Thus, the ultrasound diagnostic device is capable of performing the generation of acoustic line signals based on the subsequent transmission event so that the ultrasound image yielded as a result images the puncture needle with high visual perceptibility.

Another aspect of the present disclosure is an ultrasound diagnostic device with any structure described above, wherein the transmission event preceding the processing-target transmission event may be a most recent transmission event having been performed by using the same first group as the processing-target transmission event.

With this structure, the ultrasound diagnostic device is capable of producing ultrasounds images imaging the puncture needle with high visual perceptibility, by selecting a reception transducer element array for a processing-target transmission event based on accurate puncture needle position/inclination angle calculated based on acoustic line signals generated for a transmission event performed by using the same transmission transducer element array as the processing-target transmission event.

Another aspect of the present disclosure is an ultrasound diagnostic device with any structure described above, wherein the transmission event preceding the processing-target transmission event may include two or more transmission events having been performed after a most recent transmission event having been performed by using the same first group as the processing-target transmission event, and the receiver may extract the linear area from each of two or more sub-frame data items corresponding to the two or more transmission events.

With this structure, the ultrasound diagnostic device is capable of producing ultrasounds images imaging the puncture needle with high visual perceptibility, by being capable of selecting a reception transducer element array for a processing-target transmission event based on accurate puncture needle position/inclination angle calculated based on acoustic line signals generated for a plurality of transmission events.

Another aspect of the present disclosure is an ultrasound diagnostic device with any structure described above, further including a probe interface that, upon connection of the ultrasound probe to the ultrasound diagnostic device, acquires probe identification information identifying the ultrasound probe, wherein the receiver may specify a position and an inclination angle of the puncture needle based on the probe identification information, and selects the second group based on the position and the inclination angle.

With this structure, the ultrasound diagnostic device is capable of producing ultrasound images in which puncture needles appear with high visual perceptibility, by being capable of specifying puncture needle position/inclination angle with a simple structure incorporating a probe interface, and without having to execute processing with heavy load of detecting puncture needle position/inclination angle based on acoustic line signals.

Another aspect of the present disclosure is an ultrasound diagnostic device with any structure described above, wherein the ultrasound diagnostic device may be connectable to an operation receiver receiving input from an external source, and when the operation receiver receives input of information indicating a position and an inclination angle of the puncture needle, the receiver may select the second group based on the information.

With this structure, the ultrasound diagnostic device is capable of producing ultrasound images imaging a puncture needle appear with high visual perceptibility by being capable of specifying puncture needle position/inclination angle without a probe interface, and thus with an even simpler structure. Specifically, the ultrasound diagnostic device is capable of specifying puncture needle position/inclination angle by receiving information indicating puncture needle position/inclination angle that has been input on the operation receiver.

Another aspect of the present disclosure is an ultrasound diagnostic device with any structure described above, wherein the receiver may perform the selection of the second group for each of the transmission events based on the first group for the transmission event, the second groups for the transmission events differing from one another.

With this structure, the ultrasound diagnostic device is capable of performing reception beam forming utilizing the synthetic aperture method, where the transmission transducer element array is gradually shifted in the array direction between different transmission events, by selecting, for each transmission event, a group of transducer elements whose receive signals based on ultrasound reflection from the puncture needle have relatively high signal intensity as a reception transducer element array based on the transmission transducer element array for the transmission event.

Another aspect of the present disclosure is an ultrasound diagnostic device with any structure described above, wherein the receiver may include a delay-and-sum calculator that generates an acoustic line signal for each of the measurement points in the target area by performing delay-and-sum processing on receive signals corresponding to ultrasound reflection from the measurement point, and the delay-and-sum calculator may generate an acoustic line signal for each of the measurement points by: (i) calculating, for each of the transducer elements in the second group, a total propagation time required for transmitted ultrasound to reach the transducer element after being reflected at the measurement point, by calculating a sum of a transmission time required for the transmitted ultrasound to reach the measurement point and a reception time required for ultrasound reflection from the measurement point to reach the transducer element; (ii) calculating delay amounts for the transducer elements in the second group based on the total propagation time for each of the transducer elements in the second group; (iii) for each of the transducer elements in the second group, specifying a receive signal corresponding to the delay amount for the transducer element in the sequence of receive signals for the transducer element; and (iv) summing the specified receive signals for the transducer elements in the second group.

With this structure, the ultrasound diagnostic device is capable of generating, for each ultrasound transmission event, not only acoustic line signals based on ultrasound reflection from an area of the ultrasound main irradiation area along the central axis of the transmitted ultrasound beam, but also acoustic line signals based on ultrasound reflection from areas of the ultrasound main irradiation area other than the rather narrow area along the central axis of the transmitted ultrasound beam, and thus, improves spatial resolution and signal S/N ratio as well as enhancing the efficiency of use of transmitted ultrasound.

Further, the ultrasound diagnostic device, by utilizing the synthetic aperture method, combines acoustic line signals generated in response to different transmission events for the same measurement point. Thus, the ultrasound diagnostic device achieves the effect of performing, for multiple transmission events, virtual transmission focusing even for measurement points that are located in depths other than that of the transmission focal point, and thus further improves spatial resolution and S/N ratio.

Another aspect of the present disclosure is an ultrasound diagnostic device with any structure described above, wherein when the measurement point is deeper in the examination subject than a predetermined depth at which transmitted ultrasound focuses in the examination subject, the delay-and-sum calculator may calculate the transmission time by dividing a sum of a first distance and a second distance by velocity, and when the measurement point is shallower in the examination subject than the predetermined depth, the delay-and-sum calculator may calculate the transmission time by subtracting the second distance from the first distance and dividing the difference by velocity, where the first distance is a distance between a center position of the first group in the array direction and a beam center of transmitted ultrasound at the predetermined depth, and the second distance is a distance between the beam center and the measurement point.

With this structure, the ultrasound diagnostic device adaptively performs delay processing using the total propagation path depending upon whether the processing-target measurement point is deeper or shallower than the transmission focal point of the transmitted ultrasound. Thus, the ultrasound diagnostic device is capable of performing delay-and-sum processing focusing on every measurement point in the target area, and thus is capable of generating an acoustic line signal for every measurement point. Accordingly, the ultrasound diagnostic device is capable of generating ultrasound images with higher resolution, less noise, and higher image quality for the entirety of the ultrasound main irradiation area than conventional ultrasound images.

Another aspect of the present disclosure is an ultrasound signal processing method of: (i) repeatedly performing transmission events, each by selecting a first group of transducer elements from among a plurality of transducer elements arrayed along an array direction of an ultrasound probe and causing the first group to transmit ultrasound towards an examination subject having a puncture needle inserted therein; (ii) for each of the transmission events, generating a sub-frame data item based on ultrasound reflection received in response to the transmission event, the sub-frame data item including acoustic line signals; and (iii) combining sub-frame data items for the transmission events to generate a frame data item, the frame data item including combined acoustic line signals each being an aggregate of ones of the acoustic line signals, the ultrasound signal processing method including: performing each of the transmission events by selecting the first group and causing the first group to transmit ultrasound focusing inside the examination subject, the first group being gradually shifted in the array direction between transmission events; for each of the transmission events, selecting at least some of the plurality of transducer elements and generating a sequence of receive signals for each of the selected transducer elements based on ultrasound reflection that the selected transducer element has received from the examination subject in response to the transmission event; selecting a second group of transducer elements, the transducer elements in the second group being ones among the plurality of transducer elements whose receive signals based on ultrasound reflection from the puncture needle have high intensity; for each of the transmission events, setting a target area for generating the sub-frame data item inside a virtual area of the examination subject that receives ultrasound transmitted in the transmission event, and generating the sub-frame data item by performing, for each of a plurality of measurement points in the target area, delay-and-sum processing on receive signals, for the transducer elements in the second group, based on ultrasound reflection from the measurement point; and generating the frame data item by combining the sub-frame data items for the transmission events.

Further, another aspect of the present disclosure is a non-transitory computer-readable recording medium with the ultrasound signal processing method recorded thereon.

The ultrasound signal processing method achieves producing ultrasound images imaging a puncture needle with high visible perceptibility, with reception beam forming utilizing the synthetic aperture method.

As described up to this point, the ultrasound diagnostic device and the ultrasound signal processing method pertaining to the present disclosure achieve producing ultrasound images imaging a puncture needle with high visible perceptibility, with reception beam forming utilizing the synthetic aperture method.

### <<Supplement>>

Each of the embodiments described above should be construed as being a preferable and specific example of implementation of the technology pertaining to the present disclosure. As such, any value, any shape, any material, any constituent element, any position of any constituent element, any connection of any constituent element, any step, and any order in which any step is performed, in the embodiments, shall be construed as being a non-limiting example. Further, among the constituent elements described in the embodiments, any constituent element not recited in the independent claims, which represent the broadest concept of the present disclosure, shall be construed as a constituent element not necessarily essential but included in a preferable form of implementation of the technology pertaining to the present disclosure.

Further, in order to facilitate understanding, constituent elements described in the embodiments may be illustrated in drawings at a scale differing from their actual sizes. Further, the technology pertaining to the present disclosure shall not be construed as being limited to the embodiments, and instead, shall be construed as encompassing any modification that does not depart from the spirit and the scope of the present disclosure.

Further, the embodiments and modifications do not provide description of circuit parts and lead wires disposed on substrates in ultrasound diagnostic devices. This is since various forms of electric wiring and electric circuitry are implementable based on knowledge possessed by a skilled artisan in the present field of technology, and are not directly essential in describing the technology pertaining to the present disclosure. Further, all drawings referred to in the above are schematic drawings and may not be accurate in a strict sense.

Although the technology pertaining to the present disclosure has been fully described by way of examples with reference to the accompanying drawings, it is to be noted that various changes and modifications will be apparent to those skilled in the art. Therefore, unless such changes and modifications depart from the scope of the technology pertaining to the present disclosure, they should be construed as being included therein.

What is claimed is:

1. An ultrasound diagnostic device to which an ultrasound probe having a plurality of transducer elements arrayed along an array direction is connectable, wherein
the ultrasound diagnostic device:
(i) repeatedly performs transmission events, each by selecting a first group of transducer elements from among the plurality of transducer elements and causing the first group to transmit ultrasound towards an examination subject having a puncture needle inserted therein;
(ii) for each of the transmission events, generates a sub-frame data item based on ultrasound reflection received in response to the transmission event, the sub-frame data item including acoustic line signals; and
(iii) combines sub-frame data items for the transmission events to generate a frame data item, the frame data item including combined acoustic line signals each being an aggregate of ones of the acoustic line signals, and
the ultrasound diagnostic device comprises an ultrasound signal processing circuit operating as:
a transmitter that performs each of the transmission events by selecting the first group and causing the first group to transmit ultrasound focusing inside the examination subject, the transmitter gradually shifting the first group in the array direction between transmission events; and
a receiver that:
(i) for each of the transmission events, selects at least some of the plurality of transducer elements and generates a sequence of receive signals for each of the selected transducer elements based on ultrasound reflection that the selected transducer element has received from the examination subject in response to the transmission event;
(ii) selects a second group of transducer elements, the transducer elements in the second group being ones among the plurality of transducer elements whose receive signals based on ultrasound reflection from the puncture needle have high intensity;
(iii) for each of the transmission events, sets a target area for generating the sub-frame data item inside a virtual area of the examination subject that receives ultrasound transmitted in the transmission event, and generates the sub-frame data item by performing, for each of a plurality of measurement points in the target area, delay-and-sum processing on receive signals, for the transducer elements in the second group, based on ultrasound reflection from the measurement point; and
(iv) generates the frame data item by combining the sub-frame data items for the transmission events.

2. The ultrasound diagnostic device of claim 1, wherein
the receiver detects, from among the generated sequences of receive signals, a sequence of receive signals including a high intensity receive signal, specifies a high intensity transducer element, among the selected transducer elements, corresponding to the high intensity receive signal, and selects a group of transducer elements including the high intensity transducer element as the second group.

3. The ultrasound diagnostic device of claim 2, wherein
the receiver specifies two or more high intensity transducer elements based on different ones of the transmission events, and selects a group of transducer elements including the two or more high intensity transducer elements as the second group.

4. The ultrasound diagnostic device of claim 1, wherein
from at least one of the sub-frame data items, the receiver extracts a linear area composed of acoustic line signals with high intensity, calculates a position and an inclination angle at which the puncture needle is imaged based on the linear area, and selects the second group based on the position and the inclination angle.

5. The ultrasound diagnostic device of claim 4, wherein
the at least one of the sub-frame data items is a sub-frame data item having been generated for a transmission event preceding a processing-target transmission event.

6. The ultrasound diagnostic device of claim 5, wherein
the transmission event preceding the processing-target transmission event is a most recent transmission event having been performed by using the same first group as the processing-target transmission event.

7. The ultrasound diagnostic device of claim 5, wherein
the transmission event preceding the processing-target transmission event includes two or more transmission events having been performed after a most recent transmission event having been performed by using the same first group as the processing-target transmission event, and
the receiver extracts the linear area from each of two or more sub-frame data items corresponding to the two or more transmission events.

8. The ultrasound diagnostic device of claim 1, further comprising:
a probe interface that, upon connection of the ultrasound probe to the ultrasound diagnostic device, acquires probe identification information identifying the ultrasound probe, wherein
the receiver specifies a position and an inclination angle of the puncture needle based on the probe identification information, and selects the second group based on the position and the inclination angle.

9. The ultrasound diagnostic device of claim 1, wherein
the ultrasound diagnostic device is connectable to an operation receiver receiving input from an external source, and
when the operation receiver receives input of information indicating a position and an inclination angle of the puncture needle, the receiver selects the second group based on the information.

10. The ultrasound diagnostic device of claim 1, wherein
the receiver performs the selection of the second group for each of the transmission events based on the first group for the transmission event, the second groups for the transmission events differing from one another.

11. The ultrasound diagnostic device of claim 1, wherein
the receiver includes a delay-and-sum calculator that generates an acoustic line signal for each of the measurement points in the target area by performing delay-and-sum processing on receive signals corresponding to ultrasound reflection from the measurement point, and
the delay-and-sum calculator generates an acoustic line signal for each of the measurement points by:
(i) calculating, for each of the transducer elements in the second group, a total propagation time required for transmitted ultrasound to reach the transducer element after being reflected at the measurement point, by calculating a sum of a transmission time required for the transmitted ultrasound to reach the measurement point and a reception time required for ultrasound reflection from the measurement point to reach the transducer element;
(ii) calculating delay amounts for the transducer elements in the second group based on the total propagation time for each of the transducer elements in the second group;
(iii) for each of the transducer elements in the second group, specifying a receive signal corresponding to the delay amount for the transducer element in the sequence of receive signals for the transducer element; and
(iv) summing the specified receive signals for the transducer elements in the second group.

12. The ultrasound diagnostic device of claim 11, wherein
when the measurement point is deeper in the examination subject than a predetermined depth at which transmitted ultrasound focuses in the examination subject, the delay-and-sum calculator calculates the transmission time by dividing a sum of a first distance and a second distance by velocity, and
when the measurement point is shallower in the examination subject than the predetermined depth, the delay-and-sum calculator calculates the transmission time by subtracting the second distance from the first distance and dividing the difference by velocity, where
the first distance is a distance between a center position of the first group in the array direction and a beam center of transmitted ultrasound at the predetermined depth, and
the second distance is a distance between the beam center and the measurement point.

13. An ultrasound signal processing method of:
(i) repeatedly performing transmission events, each by selecting a first group of transducer elements from among a plurality of transducer elements arrayed along an array direction of an ultrasound probe and causing the first group to transmit ultrasound towards an examination subject having a puncture needle inserted therein;
(ii) for each of the transmission events, generating a sub-frame data item based on ultrasound reflection received in response to the transmission event, the sub-frame data item including acoustic line signals; and
(iii) combining sub-frame data items for the transmission events to generate a frame data item, the frame data item including combined acoustic line signals each being an aggregate of ones of the acoustic line signals,
the ultrasound signal processing method comprising:
performing each of the transmission events by selecting the first group and causing the first group to transmit ultrasound focusing inside the examination subject, the first group being gradually shifted in the array direction between transmission events;
for each of the transmission events, selecting at least some of the plurality of transducer elements and generating a sequence of receive signals for each of the selected transducer elements based on ultrasound reflection that the selected transducer element has received from the examination subject in response to the transmission event;
selecting a second group of transducer elements, the transducer elements in the second group being ones among the plurality of transducer elements whose receive signals based on ultrasound reflection from the puncture needle have high intensity;
for each of the transmission events, setting a target area for generating the sub-frame data item inside a virtual area of the examination subject that receives ultrasound transmitted in the transmission event, and generating the sub-frame data item by performing, for each of a plurality of measurement points in the target area, delay-and-sum processing on receive signals, for the transducer elements in the second group, based on ultrasound reflection from the measurement point; and
generating the frame data item by combining the sub-frame data items for the transmission events.
